(19) United States
(12) Patent Application Publication
(10) Pub. No.: US 2008/0226682 A1
(43) Pub. Date: Sep. 18, 2008

(54) Immunoactive Compositions for Improved Oral Delivery of Vaccines and Therapeutic Agents

(75) Inventors: David A. Brake, East Lyme, CT (US); Grant W. Vandenberg, Saint-Augustin-de-Desmaures (CA); Timothy J. Miller, Lincoln, NE (US)

Correspondence Address: FAEGRE & BENSON LLP;PATENT DOCKETING, 2200 WELLS FARGO CENTER, 90 SOUTH SEVENTH STREET, MINNEAPOLIS, MN, 55402-3901, US

(73) Assignee: PerOs Systems Technologies, Inc.

(21) Appl. No.: 12/045,791

(22) Filed: Mar. 11, 2008

Related U.S. Application Data
(60) Provisional application No. 60/894,563, filed on Mar. 13, 2007.

(51) Int. Cl. A61K 47/12; A61K 47/06

(52) U.S. Cl. 424/278.1; 514/784

(57) ABSTRACT

The present invention concerns methods and compositions for improved oral delivery of bioactive agents, such as vaccines. In preferred embodiments, the compositions comprise at least one lectin, saponin, polyunsaturated fatty acid and/or isoflavone. In further embodiments, the compositions may further comprise at least one protease inhibitor, buffer and/or surfactant. In more preferred embodiments, the lectins, saponins, fatty acids, isoflavones and/or protease inhibitors may be derived from extracts, homogenates, finely ground powders or derivatives of plant or animal material, such as beans, nuts, peas, fish meal or krill. The relative amounts of various naturally occurring materials contained in the compositions may be selected to optimize the concentrations of one or more lectins, saponins, polyunsaturated fatty acids and/or isoflavones. The compositions are of use for oral delivery of a wide variety of bioactive agents, particularly protein or peptide based agents.

### BACKGROUND

1. Field of the Invention

The present invention concerns methods and compositions for oral delivery of bioactive agents. In particular embodiments, the compositions may comprise one or more lectins, isoflavones, polyunsaturated fatty acids, saponins, protease inhibitors, surfactants, buffers and bioactive agents. In preferred embodiments, one or more of the components of the compositions may be derived from monocot or dicot plant sources, such as extracts, homogenates or ground powders of beans, peas or nuts. Alternatively, such components may also be found in other plant parts or in non-plant material, such as fish meal or krill. In more preferred embodiments, the type and/or amount of naturally occurring ingredients, such as homogenates or fine ground powders of beans, peas, nuts, plant parts, fish meal or krill used in the claimed compositions may be selected to optimize the content of specific lectins, isoflavones, polyunsaturated fatty acids, saponins and/or protease inhibitors present in the final composition. The methods and compositions are effective for oral delivery of a wide variety of bioactive agents to a wide range of subjects.

2. Description of Related Art

Administration of bioactive agents, such as drugs, vaccines, hormones and therapeutic peptides, may occur by various routes. Parenteral injection (intravenous, intramuscular, subcutaneous, etc.) is often used. However, parenteral administration is labor-intensive and time consuming when large numbers of subjects must be treated, as in fish farms, cattle feedlots and similar operations. Further, compositions for parenteral administration often must be kept refrigerated, with limited shelf life and spoilage problems in areas where refrigerated distribution and storage infrastructure is deficient, as in many developing or underdeveloped countries. Parenteral administration to humans also requires the availability of trained personnel to perform the injection. Parenteral injection can also cause bruising or bleeding at the injection site as well as inflammatory site reactions that result in condemnation of the meat at the slaughter house or processing plant.

Oral administration of bioactive agents may therefore be preferred. However, oral administration of a number of classes of agents is limited by poor absorption, degradation by gastric and intestinal enzymes or instability of the agent in aqueous solutions generally and in the low pH environment of the stomach in particular. This is especially problematic for delivery of protein or peptide bioactive agents, which at present are primarily administered parenterally. However, other types of bioactive agents may exhibit similar problems when orally administered.

A number of attempts have been made to develop compositions and methods for oral delivery of bioactive agents. Kidron (U.S. Pat. No. 4,579,730) proposed the oral administration of enterocoated compositions containing insulin, bile acids or bile salts and protease inhibitors. Desai (U.S. Pat. No. 5,206,219) suggested the oral administration of enteric coated compositions containing proteinaceous medicaments, polyol solvents, lipids and protease inhibitors. Fasano (U.S. Pat. No. 5,665,389) suggested the oral administration of therapeutic agents with a zonula occludens toxin, such as purified Vibrio cholera zonula occludens toxin. However, such earlier attempts focused on the use of purified or semipurified ingredients to achieve interaction with specific coatings or compounds. In the context of veterinary, aquaculture or livestock use, such purified or semipurified components may render the compositions too expensive for practical oral administration to non-human subjects, nor in earlier attempts have the components of bean powders been identified from natural ingredients that can provide the environment for complex antigen mixtures.

Another approach to compositions for oral delivery of bioactive agents was disclosed in U.S. Patent Application Publication Nos. 20030118547 and 20050175724. Those applications discussed three major features of the compositions: (1) The use of anti-protease derived from biological components, such as ground bean extracts or ovalbumin. (2) The use of neutralizing agents, such as buffers, to neutralize stomach pH. (3) The use of uptake-increasing agents, such as detergents, to improve absorption across the intestinal wall. While such compositions provided advantages over earlier methods for oral delivery, additional components found in naturally occurring bean materials and powders, such as lectins, saponins, isoflavones and polyunsaturated fatty acids, may provide further advantages for oral delivery of bioactive agents. Such additional components of naturally occurring materials and their effects on oral delivery of bioactive agents, including physiological and immune sensitization activities, have not been investigated prior to the instant disclosure. Such components may have the ability to greatly enhance targeted oral delivery, stability and uptake of various bioactive agents, for example based on their combinations, concentrations and specificities for cell receptors, cell membranes and cellular communication pathways involved in biological activity, and/or immune sensitization in the target species of interest.

### SUMMARY OF THE INVENTION

The present invention fulfills an unresolved need in the art by providing methods and compositions for oral delivery of bioactive agents. In particular embodiments, the compositions may comprise one or more lectins, isoflavones, polyunsaturated fatty acids, saponins, protease inhibitors, surfactants, buffers and/or bioactive agents. In preferred embodiments, one or more of the components of the compositions may be derived from plant sources, such as extracts, homogenates or ground powders of beans, peas or nuts. Alternatively, the components may be derived from other plant parts or from non-plant sources, such as fish meal or krill. In various embodiments, the types and amounts of different naturally occurring materials, such as homogenates of beans, peas, nuts, other plant parts, fish meal or krill, may be selected to optimize (e.g, by using finely ground materials, varying concentration ranges and/or ratios of materials) the content of specific lectins, isoflavones, polyunsaturated fatty acids, saponins and/or protease inhibitors present in the final composition. Such compositions may vary depending on the type of bioactive agent to be administered, the target species to whom the agent is to be administered, the disease or condition that is addressed by such administration, and the desired effect of the bioactive agent on the target species.

Certain embodiments may concern the use of finely ground material, such as finely ground beans or extracts of finely ground materials. Such finely ground materials have a very high surface to mass ratio, providing improved absorption and bioavailability of the contents of the ground material. Compared with crude mortar and pestle type grinding, the use of finely ground powders, generated for example with a powder mill and small mesh screen, provide increased activities of anti-proteases, lectins, saponins and other components found in naturally occurring plant material. The particle size of ground plant or animal materials used and the amounts of such materials in the final composition may be varied to optimize the oral availability, length of time during which absorption occurs and/or the immunological or other characteristics of orally delivered bioactive agents. In various embodiments, the particle size of finely ground material may be 0.25 to 5 mm, 0.5 to 2.5 mm, 1 to 5 mm, 0.1 to 1.0 mm, or any combination of such ranges. Such finely ground powders may be easily stored and/or mixed after grinding.

Bioactive agents to be delivered by oral administration using the claimed methods and compositions may include, but are not limited to, drugs, pharmaceuticals, toxins, anti-cancer agents, anti-inflammatory agents, antibiotics, antifungals, antiviral agents, anti-parasitic agents, vaccines, adjuvants, antigens, hormones, growth factors, cytokines, chemokines, immunomodulators, interferons, interleukins, hematopoietic factors, coagulation factors, anti-angiogenic factors, pro-apoptosis factors, neurotransmitters, neuromodulators, enzymes, agonists, antagonists, antibodies, antibody fragments, fusion proteins, proteins, polypeptides, peptides, nucleic acids, lipids, polysaccharides, carbohydrates or steroids. In certain preferred embodiments, the bioactive agent may be a protein or peptide based agent.

Lectins of use may include, but are not limited to, Con A, L4, L3E1, SBL, PNL, BBL, PHA, PHA-E, PHA-L, PSA, SBA, PNA, LCA, LOA, LBL, jacalin or WGA. In preferred embodiments, the lectins of use may be present in an extract, homogenate, finely ground powder, or other derivative of plant matter, such as beans, peas or nuts. However, other sources of lectins are known and in alternative embodiments, other natural sources such as different plant parts or non-plant materials (e.g., fish meal, krill) may be utilized. In more preferred embodiments, the relative proportions of plant or non-plant extracts, homogenates, finely ground powders or derivatives may be selected to optimize the content of one or more lectins of use in the composition for oral delivery of bioactive agents. Various lectins and their properties are discussed in more detail below.

Isoflavones of use may include, but are not limited to, gentisein, daidzein, biochanin, biochanin A, formononctin, glycitein or formononetin. In preferred embodiments, the isoflavones of use may be present in an extract, homogenate, finely ground powders or other derivative of plant matter, such as beans, peas, nuts or other plant parts. In more preferred embodiments, the relative proportions of plant extracts, homogenates, finely ground powder or derivatives may be selected to optimize the content of one or more isoflavones of use in the composition for oral delivery of bioactive agents. Isoflavones and their properties are discussed in more detail below. Further details on isoflavones of possible use may be found, for example, in U.S. Pat. Nos. 5,679,806 and 6,146,668.

A number of polyunsaturated fatty acids are known and any such known polyunsaturated fatty acids may be of use in the disclosed methods and compositions. Polyunsaturated fatty acids of use may include, but are not limited to, soybean n-3, soybean n-6, kidney bean n-3, kidney bean n-6 and lima bean n-6 polyunsaturated fatty acid. In preferred embodiments, the polyunsaturated fatty acids of use may be present in an extract, homogenate, finely ground powder or other derivative of plant matter, such as beans, peas, nuts or other plant parts. In more preferred embodiments, the relative proportions of plant extracts, homogenates, finely ground powders or derivatives may be selected to optimize the content of one or more polyunsaturated fatty acids of use in the composition for oral delivery of bioactive agents. Polyunsaturated fatty acids and their properties are discussed in more detail below.

Saponins of use may include, but are not limited to, soyasaponin A(1), soyasaponin A(2), soyasaponin I, soyasaponin B, deacetylated soyasaponin, acetylated soyasaponin, soyasaponin II, soyasaponin III and soyasapogenol B monoglucuronide. In preferred embodiments, the saponins of use may be present in an extract, homogenate, finely ground powders, or other derivative of plant matter, such as beans, peas, nuts or other plant parts. In more preferred embodiments, the relative proportions of plant extracts, homogenates, finely ground powders, or derivatives may be selected to optimize the content of one or more saponins of use in the composition for oral delivery of bioactive agents. Saponins and their properties are discussed in more detail below.

In various embodiments, the protease inhibitor activity can be increased from powderized beans and used to stabilize the ‘active’ agent as well as carrier feed and may comprise any protease inhibitor known in the art, such as albumen, ethylenediamine tetraacetate (EDTA), alpha-1-antitrypsin, proteosomes, aprotinin (Trasilol™), pentamidine isethionate, antipain, tosylamide-phenylethyl-chloromethyl ketone (TPCK), phenylmethyl sulfonyfluoride (PMSF), pepstatin, trypsin inhibitor, acetone, alcohols, guanidium, α2-macroglubulin, TLCK, chelating agents, iodoacetate, Zn⁺², antithrombin III, leupeptin, potato carboxypeptidase inhibitor and chymostatin. Such inhibitors may be found in a wide variety of biological materials, including both plant and animal sources. In preferred embodiments, the protease inhibitor may be contained in an extract or homogenate or finely ground powders of beans or oilseeds. However, protease inhibitors from other plant sources or non-plant sources may also be of use in the claimed methods and compositions. In more preferred embodiments, the protease inhibitor(s) and other components may be used in the form of fine powders to stabilize the carrier formulation, increase availability, absorption and/or stabilize the active conformation of the bioactive agent, for example by increasing release of protease inhibitors or lectins from the ground plant matter. Increased stability of bioactive agents in the formulations may occur in the gastrointestinal system after oral ingestion, as well as in storage before ingestion. For example, binding of lectins to vaccines or other protein ingredients may stabilize their conformation and prevent or decrease degradation.

It is anticipated that any buffering agent known in the art may be utilized, including but not limited to Tris-HCL, carbonate/bicarbonate, malate, pyridine, piperazine, cacodylate, succinate, MES, citrate, maleate, bis-tris, phosphate, ethanolamine, ADA, ACES, PIPES, MOPSO, imidazole, BES, MOPS, HEPES, TES, MOBS, DIPSO, TAPSO, HEPPSO, POPSO, tricine, hydrazine, glycylglycine, EPPS, HEPPS, BICINE, HEPBS, TAPS, AMPD, TABS, AMPSO, taurine, borate, CHES, AMP, glycine, ammonium hydroxide, CAPSO, methylamine or CAPS.

A variety of surfactants are known and may be used, although non-denaturing surfactants are preferred. Surfactants may generally be categorized as anionic, cationic, zwiterionic or neutral. Examples of surfactants include fatty acids, alkyl benzene sulfonate, Brij, CHAPS, CHAPSO, CTAB, CPC, POEA, BAC, BZT, dodecyl betaine, dodecyl dimethylamine oxide, dodecyl-β-D-maoltoside, cocamidopropyl betaine, coco ampho glycinate, octyl glucoside, octyl thioglucopyranoside, decyl maltoside, alkyl poly(etheylene oxide), sodium cholate, sodium deoxycholate, Triton X-100, Triton X-114, NP-40 and Tween.

In various embodiments, the saponins, lectins, polyunsaturated fatty acids, isoflavones and/or protease inhibitors may be derived from plant materials. In preferred embodiments, plant material of use may be an extract, homogenate, finely ground powder or derivative of one or more beans, peas or nuts. However, other plant parts or non-plant sources may also be utilized. Non-limiting examples of plant material of use include, but are not limited to, soybean, lima bean, fava bean, kidney bean, red kidney bean, broad bean, jequirity bean, jack bean, small pea, sweet pea, Rosemary pea, lentil, vetch and peanut. In more preferred embodiments, the relative amounts of such extracts, homogenates, finely ground powders or derivatives from different plant and/or non-plant materials may be selected to optimize the content of particular lectins, saponins, polyunsaturated fatty acids, isoflavones and/or protease inhibitors. Such optimization may be of use to modulate or modify the stability, oral delivery, absorption, bioavailability, immunogenic properties and/or efficacy of the bioactive agent. In various embodiments, the compositions and methods may be further optimized for oral delivery of bioactive agents to a selected subject species, such as an animal, mammal, human, fish, trout, salmon, carp, tilapia, catfish, shrimp, crab, lobster, abalone, snail, bivalve, oyster, mussel, clam, bird, chicken, duck, cow, buffalo, elk, deer, antelope, moose, caribou, pig, sheep (ovine), goat, dog, cat, horse, donkey, mule, alpaca or llama.

In certain embodiments, the compositions and methods may be utilized to treat or prevent disease associated with pathogenic agent infection. For examples, the compositions may be of use for oral vaccine delivery to immunize subjects against pathogen infection. Alternatively, the compositions may be of use for oral delivery of antibiotics, antiviral, antifungal, antiparasitic or other agents. The type of pathogen to be treated or vaccinated against is not limiting, but may include any of the following.

Actinobacillus spp.

Actinomyces spp.

Adenovirus (types 1, 2, 3, 4, 5 et 7)

Adenovirus (types 40 and 41)

Aerococcus spp.

Aeromonas salmonicida

Ancylostoma duodenale

Angiostrongylus cantonensis

Ascaris lumbricoides

Ascaris spp.

Aspergillus spp.

Bacillus anthracis

Bacillus cereus

Bacteroides spp.

Balantidium coli

Bartonella bacilliformis

Blastomyces dermatitidis

Bluetongue virus

Bordetella bronchiseptica

Bordetella pertussis

Borrelia burgdorferi

Branhamella catarrhalis

Brucella spp.

B. abortus

B. canis,

B. melitensis

B. suis

Brugia spp.

Burkholderia mallei

Burkholderia pseudomallei

Campylobacter fetus subsp. fetus

Camplylobacter jejuni

C. coli

C. fetus subsp. jejuni

Candida albicans

Capnocytophaga spp.

Chlamydia psittaci

Chlamydia trachomatis

Citrobacter spp.

Clonorchis sinensis

Clostridium botulinum

Clostridium difficile

Clostridium perfringens

Clostridium tetani

Clostridium spp.

Coccidioides immitis

Colorado tick fever virus

Corynebacterium diphtheriae

Coxiella burnetii

Coxsackievirus

Creutzfeldt-Jakob agent, Kuru agent

Crimean-Congo hemorrhagic fever virus

Cryptococcus neoformans

Cryptosporidium parvum

Cytomegalovirus

Dengue virus (1, 2, 3, 4)

Diphtheroids

Eastern (Western) equine encephalitis

Ebola virus

Echinococcus granulosus

Echinococcus multilocularis

Echovirus

Edwardsiella tarda

Entamoeba histolytica

Enterobacter spp.

Enterovirus 70

Epidermophyton floccosum,

Microsporum spp. Trichophyton spp.

Epstein-Barr virus

Escherichia coli, enterohemorrhagic

Escherichia coli, enteroinvasive

Escherichia coli, enteropathogenic

Escherichia coli, enterotoxigenic

Fasciola hepatica

Francisella tularensis

Fusobacterium spp.

Gemella haemolysans

Giardia lamblia

Giardia spp.

Haemophilus ducreyi

Haemophilus influenzae (group b)

Hantavirus

Hepatitis A virus

Hepatitis B virus

Hepatitis C virus

Hepatitis D virus

Hepatitis E virus

Herpes simplex virus

Herpesvirus simiae

Histoplasma capsulatum

Human coronavirus

Human immunodeficiency virus

Human papillomavirus

Human rotavirus

Human T-lymphotrophic virus

Influenza virux

Infectious pancreatic necrosis virus

Junin virus/Machupo virus

Klebsiella spp.

Kyasanur Forest disease virus

Lactobacillus spp.

Legionella pneumophila

Leishmanis spp.

Leptospira interrogans

Listeria monocytogenes

Lymphocytic choriomeningitis virus

Marburg virus

Measles virus

Micrococcus spp.

Moraxella spp.

Mycobacterium spp.

Mycobacterium tuberculosis, M. bovis

Mycoplasma hominis, M. orale, M. salivarium, M. fermentans

Mycoplasma pneumoniae

Naegleria fowleri

Necator americanus

Neisseria gonorrhoeae

Neisseria meningitidis

Neisseria spp.

Nocardia spp.

Norwalk virus

Omsk hemorrhagic fever virus

Onchocerca volvulus

Opisthorchis spp.

Parvovirus B19

Pasteurella spp.

Peptococcus spp.

Peptostreptococcus spp.

Piscirickettsia salmonis

Plesiomonas shigelloides

Powassan encephalitis virus

Proteus spp.

Pseudomonas spp.

Rabies virus

Respiratory syncytial virus

Rhinovirus

Rickettsia akari

Rickettsia prowazekii, R. canada

Rickettsia rickettsii

Ross river virus/O'Nyong-Nyong virus

Rubella virus

Salmonella choleraesuis

Salmonella paratyphi

Salmonella typhi

Salmonella spp.

Schistosoma spp.

Scrapie agent

Serratia spp.

Shigella spp.

Sindbis virus

Sporothrix schenckii

St. Louis encephalitis virus

Murray Valley encephalitis virus

Staphylococcus aureus

Steptobacillus moniliformis

Streptococcus agalactiae

Streptococcus faecalis

Streptococcus pneumoniae

Streptococcus pyogenes

Streptococcus salivarius

Taenia saginata

Taenia solium

Toxocara canis, T. cati

Toxoplasma gondii

Treponema pallidum

Trichinella spp.

Trichomonas vaginalis

Trichuris trichiura

Trypanosoma brucei

Ureaplasma urealyticum

Vaccinia virus

Varicella-zoster virus

Venezuelan equine encephalitis

Vesicular stomatitius virus

Vibrio cholerae, serovar 01

Vibrio parahaemolyticu

Wuchereria bancrofti

Yellow fever virus

Yersinia enterocolitica

Yersinia pseudotuberculosis

Yersinia pestis

### BRIEF DESCRIPTION OF THE DRAWINGS

The following drawings form part of the present specification and are included to further demonstrate certain aspects of particular embodiments of the invention. The embodiments may be better understood by reference to one or more of these drawings in combination with the detailed description presented herein.

FIG. 1. Effect of F1 and F2 compositions on stability of Newcastle disease virus (NDV) at room temperature (R.T.).

FIG. 2. Effect of Oralject™ F1 and F2 compositions on stability of Newcastle disease virus (NDV) at 2-7° C.

FIG. 3. Western blot of NDV antigens extracted from Oralject™ F1 and F2 compositions using Tween 80 extraction buffer.

FIG. 4. Western blots of NDV antigens extracted from Oralject™ F1 and F2 compositions using Triton X-100 extraction buffer.

FIG. 5. Effect of various detergent compositions on NDV antigen extraction in Oralject™ F1 and F2 compositions.

FIG. 6. Effect of F3, F4, F5, and F6 purified bean components on NDV antigen extraction in the absence or presence of Triton X-100 extraction buffer.

FIG. 7. Proliferative Effect of Purified Lectins on Peripheral Blood Mononuclear Cells (PBMC).

FIG. 8. Effect of PUFA Pretreatment on LPS Induced NO Generation

FIG. 9. Effect of Isoflavones on PHA Induced IFN-Gamma Production in Calf PBMC.

FIG. 10. Effect of Isoflavones on LPS-Induced NO Generation.

FIG. 11. Effect of Saponins on LPS-Induced NO Generation.

FIG. 12. Intestinal NDV-specific IgG response following oral immunization with Oralject™ F1 and F2 NDV vaccines.

FIG. 13. In vitro activity of the buffered anti-protease solution of the present invention.

FIG. 14. Blood glucose level as a function of time for each treated group of mice.

FIG. 15 Lack of effect of liquid formulas on the survivability of Lactobacillus acidophilus R052 at 4° and 25° C.

FIG. 16. Survivability of Lactobacillus acidophilus R052 (expressed as log CFU/G) in the gastrointestinal simulator model TIM-1

FIG. 17. Efficacy of a solid formulation to orally deliver a gram-negative acting antibiotic to aquatic species.

FIG. 18. Efficacy of a solid formulation to orally deliver a gram-positive acting antibiotic to aquatic species.

FIG. 19. Efficacy of a solid formulation to orally deliver a viral vaccine to aquatic species.

### DESCRIPTION OF ILLUSTRATIVE EMBODIMENTS

### Definitions

As used herein, “a” or “an” may mean one or more than one of an item.

As used herein, the terms “and” and “or” may be used to mean either the conjunctive or disjunctive. That is, both terms should be understood as equivalent to “and/or” unless otherwise stated.

As used herein, a “bioactive agent” refers to any chemical, molecule, composition, complex, aggregate or formulation that produces a physiological and/or therapeutic effect when administered to a subject. Such agents may include, but are not limited to, drugs, pharmaceuticals, toxins, anti-cancer agents, cytotoxic agents, anti-inflammatory agents, antibiotics, antifungals, antiviral agents, anti-parasitic agents, vaccines, adjuvants, antigens, hormones, growth factors, cytokines, chemokines, immunomodulators, interferons, interleukins, hematopoietic factors, coagulation factors, anti-angiogenic factors, pro-apoptosis factors, neurotransmitters, neuromodulators, enzymes, agonists, antagonists, antibodies, antibody fragments, fusion proteins, proteins, polypeptides, peptides, nucleic acids, lipids, polysaccharides, carbohydrates and steroids.

As used herein, the term “about” means within plus or minus ten percent (10%) of a value. For example, “about 100” would include any number between 90 and 110. The skilled artisan will realize that where a range of values is indicated, only those numbers within a permissible range are intended. For example, the skilled artisan would understand that “about 99% by weight” would not be intended to include values greater than 100% by weight.

### Compositions for Oral Delivery

Additional details concerning certain aspects of compositions for oral delivery of bioactive agents are disclosed in U.S. Patent Application Publication Nos. 20030118547, filed Jan. 25, 2001, and 20050175724, filed Apr. 8, 2003, and U.S. Provisional Patent Application No. 60/774,271, filed Feb. 17, 2006.

Compositions disclosed in the three applications above are comprised of specific ingredients that allow for improved intestinal targeted delivery of bioactive agents, such as vaccine antigens, and improved stimulation of host innate and adaptive immune responses in piscine, avian and mammalian species. The compositions may comprise: i) a neutralizing agent to increase gastrointestinal pH and reduce antigen denaturation, ii) anti-protease inhibitors to prevent enzymatic degradation of antigen, and iii) an uptake increasing agent to facilitate intestinal adsorption of antigen for subsequent immune recognition and response. In the disclosed compositions, legume-derived inhibitors from soybeans (Glycine max), kidney beans (Phaseolus vulgaris), and lima beans (Phaseolus limanis) serve as sources of anti-protease inhibitors for digestive enzymes.

However, legumes and other plants, or even animal materials, also contain other important molecules that affect delivery targeting, absorption and immunomodulator functions in the host intestinal system that positively effect oral vaccine antigenicity and efficacy and drug delivery. The important contributory role of these bean-, plant- or animal-derived ingredients, their concentrations, and relative combinatorial ratios on host immune function has not been described to date in the context of vaccine antigen (or immune stimulatory bioagents) for stability, formulation, adsorption or delivery. Such molecules as lectins, saponins, polyunsaturated fatty acids and/or isoflavones, which may be derived from plant materials such as beans, peas or nuts, other plant materials or non-plant sources may be selected for optimal oral delivery of bioactive agents, such as vaccines, and incorporated into the compositions in various concentrations and ratios for use in medical, veterinary, aquaculture and/or agricultural applications.

In preferred embodiments, the disclosed ingredients for the claimed compositions and methods may be obtained from “seed” components of plants, such as beans or peas. However, such ingredients may also be obtained from other parts of plants, or even in some cases from non-plant materials, such as fish meal or krill.

### Probiotics

Probiotics are dietary supplements containing potentially beneficial bacteria or yeast. Although the field has existed for many years, a renewed interest in the use of natural products for human or animal therapy has resulted in the rapid recent development of probiotics, either alone or in conjunction with other types of therapy.

The rationale behind probiotics is that the body contains a natural intestinal flora, comprising a variety of endemic bacteria, yeast and other organisms. Elements of this flora may include Lactobacillus, Bifidobacerium, Lactococcus, Streptococcus, Clostridium, Eubacterium, Fusobacterium, Peptococcus, Peptostreptococcus, Saccharomyces and Bacteroides. This endemic intestinal flora may play an important role in inflammation, mucosal immune response and the physical environment of the gastrointestinal tract.

Various events may negatively impact the intestinal flora, with antibiotic therapy a major factor. However, other types of events, such as aging, disease, stress, diet, alcohol consumption or other factors may also affect the makeup of endemic intestinal organisms. An underlying principal of probiotic therapy is that periodic supplementation of the natural intestinal flora with externally delivered microorganisms may assist in reestablishing a healthy balance of intestinal microorganisms. Such a balance may assist in inhibiting or preventing intestinal colonization by other, more pathogenic types of bacteria and other microorganisms. Many probiotics are present in natural sources such as lactobacillus in yogurt and other foods.

In addition to their effects on the microecology of the intestinal flora, probiotics may have immunomodulatory, antioxidant and other effects as well, that may impact orally delivered bioactive agents. Probiotics have demonstrated anti-mutagenic effects, thought to be due to their ability to bind to carcinogenic heterocylic amines in cooked meat. Probiotics are also reported to protect against colon cancer in rodents, possibly by decreasing the activity of β-glucuronidase in generating carcinogens. Lower rates of colon cancer appear to be correlated with higher consumption of fermented dairy products. Other health effects asserted for probiotics include lower blood cholesterol, lower blood pressure, improved immune function, preventing infections, reducing inflammation and improving mineral absorption.

To support probiotic therapies, it is likely that large numbers of viable microorganisms are required to be delivered to the large intestine. One major technical hurdle is to assure that viable bacteria reach the large intestine, as these organisms are largely destroyed during transit through the stomach and intestine. The instant compositions may be of use to provide more effective delivery of probiotic agents to the intestine.

In addition, the combined use of bacteria, phage, virus or other cells that can convert target ligands to inert or active compounds is a synergistic effect that may be utilized to expand the formulation versatility of compositions for oral delivery of bioactive agents described herein, with modified delivery by the oral route. The interactive effect of probiotic agents with other components, such as lectins, isoflavones, fatty acids, anti-proteases or saponins, has not been previously characterized.

### Immunoactive Plant Ingredients

There are four main components in Fabaceae sensu lato (legumes) that possess immunoactive properties: a) lectins, b) isoflavones, c) polyunsaturated fatty acids, and d) soyasaponins. As discussed above, some of these components may also be obtained from other sources. For example, PHA-type lectins from fish meal or krill may provide the same benefits as lectins from legume sources. In the discussion below, where the component source is described within the context of legumes, the skilled artisan will realize that other plant sources or even animal sources of the same or similar components may also be utilized.

### Lectins

Lectins are water soluble, carbohydrate-binding proteins found at moderately high levels in the seeds of almost 1000 different plants, and are most abundant (e.g., up to 1.5-3% of total protein content) in legumes such as soybean, kidney bean and faba beans. Seafood is the second most common source of lectins and in certain embodiments it is contemplated that fish meal, krill or other seafood sources may provide PHA-type ligands and/or other lectins. The skilled artisan will realize that in general, biological agents such as cells, parasites, bacteria, viruses, etc. have cell receptors that either contain carbohydrate or that have lectin, isoflavone, PFA and/or saponin components that can either act to induce physiological/immunological/endocrinological activities through signal transduction; enhance, up regulate or down regulate activities associated with growth or secretion; or stabilize the biological agent by inducing cryptic, dormant or inactive status of the cell or organism. In various embodiments, the concentrations of these compounds in different sources and the composition of multimeric components, especially lectins, may cause different formulations to affect different biological activities. The opportunity to utilize these compounds, from naturally occurring, partially purified or unpurified sources such as plant homogenates, ground bean powders, fish meal or krill , at varying concentrations, pH levels, ionic strength, surfactants, etc. has not previously been recognized in the field of oral delivery of physiologically active agents such as vaccines.

Most lectins are multimeric, comprised of non-covalently associated identical (e.g, Con A) or different (e.g., L4, L3E1) subunits. The multimeric structure can confer cell agglutination, and although many lectins test positive for cell agglutination, some may bind cells and not cause agglutination.

Lectins are generally heat labile, but considerable amounts remain after cooking (Ryder, S. D., 1992). Following ingestion, lectin activity is retained during passage through the gastrointestinal tract (Kilpatrick, D. C., 1985).

Lectins that bind to the sugar motif N-acetyl-D-galactosamine tend to stimulate intestinal cells (Ryder, S. D., et al., 1992; Koninkx, J. F., 1992). These include soybean (Glycine max) lectin (SBL), which binds to N-acetyl-D-galactosamine and D-galactose, and peanut (Arachis hypogaea) lectin (PNL), which binds to D-galactose-β_{—}1,3-N-acetyl-D-galactosamine. In contrast, lectins that bind to mannose or glucose tend to have no effects or even inhibit processes such as proliferation (Koninkx, J. F., 1992). Broad bean (Vicia faba) lectin (BBL) is a member of this group.

Plant lectins are antigenic, stimulate IgG antibodies and possess a diverse array of immunobiological activities. Lectins that are stimulatory for leukocytes are often referred to as mitogens, based on their ability to induce (mitotic) cell division or cell proliferation in a mature quiescent immune cell that does not normally divide (Lis, H., 1977).

Lectin mitogenic activity is historically defined by its effect on murine or human immune cells, rather than immune cells from other species. In addition, lectin tests are most commonly conducted in vitro using peripheral blood lymphocytes (PBLs), peripheral blood mononuclear cells (PBMCs), lymph node lymphocytes or splenic lymphocytes, versus intestinal lymphocytes. Since lectins recognize specific sugar ligands, lymphocytes which differ in their surface carbohydrate composition will differ in their responsiveness to the same lectin. Consequently, the lymphocyte tissue source and species origin are important criteria in characterization of lectin mitogen activity. Some lectins may be mitogenic for lymphocytes, whereas other lectins can be inhibitory for immune function. In the methods disclosed below, the effect of different lectins and/or differing lectin concentrations on lymphocyte and/or immune system function from various aquatic and terrestrial species may be assayed using standard mitogenic, binding and other known tests, for example with human or murine lymphocytes. The effect of other components, such as saponins, polyunsaturated fatty acids or isoflavones, and their ability to interact with lectins to modify their effect on immune function, may also be determined using isolated cells in vitro, and/or in vivo assays.

As discussed above, lectins of use may be obtained from either plant or non-plant sources. Further, the activities of lectins in the final composition may be affected by their physical environment, such as pH, ionic strength, presence and concentrations of surfactants, particle size of ground plant or animal material, etc. or by other bioactive components of the composition, including interactions with different lectins (e.g. competitive binding)

The red kidney bean, Phaseolus vulgaris, was the first legume shown to possess a mitogenic lectin (PHA) (Nowell, 1960) and several mitogenic lectins from Leguminosae have since been discovered (Table 1). PHA and Con A are the two most commonly used lectins for in vitro lymphocyte activity studies and both possess mitogenic activity on fish and chicken lymphocytes.

Lectins can be classified according to their carbohydrate specificity (Table 1). Some lectins will bind only to structures containing mannose or glucose residues, while others may recognize only galactose residues. Some lectins require that the specific sugar is located in a terminal non-reducing position in the oligosaccharide, while others can bind to sugars within the oligosaccharide chain. In addition, sugar specificity is not an indicator of mitogenicity and lectins do not readily segregate into mitogenic and non-mitogenic classes. In some instances, lectins require pre-treatment with neuraminidase for mitogenic effect (e.g., SBA on mouse lymphocytes; PNA on rat and human lymphocytes). As discussed above, lectins of different binding specificities may be utilized in different target species or for different purposes within the same species. Binding specificities of many lectins are known and those of others may be determined by standard binding assays known in the art, such as affinity column chromatography, microtiter well binding assay, dot or slot blot, magnetic bead binding assays, microarray assay and other such well-known techniques. In some cases, multiple lectins of different specificities and/or concentrations and/or relative ratios may be used. In preferred embodiments, the type and concentration of lectins in the final composition may be determined by the source and amount of plant and/or animal products utilized in the mix. Lectin number, type, specific concentration, and relative ratio to other lectins are used to optimize oral delivery of bioactive agents.

In addition to the source and binding specificity, lectin concentration can also determine the level of cell proliferation achieved. In general, in vitro studies suggest that low lectin concentrations are stimulatory and high concentrations are inhibitory. Structurally, many lectins are multimeric and often occur in isoforms that can influence biologic activity. For example, monomeric PNA is mitogenic whereas the dimer is not. PHA-L is more potent as a tetramer than in its dimeric form. The relative mitogenic activity of monomeric and oligomeric forms of many other plant lectins has not been studied.

A mitogenic lectin may lead to increased antibody production by B cells and increased cytokine and chemokine production by T cells. ELISA and RT-PCR assays may be used to determine the secretion profile induced by specific lectins. A summary of the properties of exemplary lectins present in bean extracts is presented below.

Soybean (Glycine max) Lectin (SBA)

Biochemistry. SBA is a tetrameric (120 kD) D-galactose/D-GalNAc-specific lectin containing one oligomannose-type chain monomer. Native SBA consists of at least five isolectins, three of which (SBA-I, -II, and -III) have been purified and characterized. The pI of the SBA isolectins ranges between pH 6.7 and 7.0. SBA maintains a high degree of stability due to oligomerization. The monomeric species (approximately 28-29.5 kDa) is only found at pH<2.0. SBA is observed by SDS-PAGE and Western blots from dehulled solvent-extracted soybean meal (DSSM), full-fat soybean meal (FFSM) and aqueuous extracts of feed formulations (Buttle, L. G., 2001). SBA carbohydrate-binding and agglutinating activity is destroyed following heat treatment at 100° C. for 5 min. In addition, urease activity is highly correlated with SBA lectin activity, suggesting that urease activity is a useful measurement to monitor lectin activity in commercial soybean meal. The effect of varying the concentrations and/or physical environment of SBA and other lectins has not yet been evaluated within target species of animals for their effect on oral delivery of bioactive agents. Nor has the effect of combination with various other active ingredients such as saponins, polyunsaturated fatty acids or isoflavones been examined.

Intestinal Bioactivity. Soybean derived SBA binds chicken enterocytes and causes hyperplasia and dysplasia of the duodenum intestinal epithelium and intestinal villi atrophy. This activity may play a role in the pathogenesis of runting and stunting syndrome in broilers fed high soybean diets. Similarly, SBA binds to the intestinal epithelium of Atlantic salmon and rainbow trout and has a contributory role in pathological changes associated with fish feeds containing high levels of soybean proteins. For example, fish fed a diet with a high level of DSSM (60% of diet) or pure SBA in proportional quantities to that found in the total protein in full-fat soybean meal exhibited pathological disruption of the intestinal tract (Buttle, L. G., 2001). These published articles conclude that the soybean lectin SBA has a deleterious effect on intestinal physiology and do not comtemplate or suggest that various lectins at various concentrations can be used to modulate oral delivery of bioagents. For example, although SBA many cause a pathological disruption in diet, it may be the very feature to allow antigen delivery across the basal membrane or within the lamina propria to stimulate intestinal immune or physiological responses. Diet supplementation in rats at >2.0 mg SBA/g body weight causes hyperplastic and hypertrophic growth of the small intestine and pancreas and causes a significant reduction in weight gain. SBA binds to the intestinal club cells, bile duct epithelial cells and renal tubule epithelial cells in the flat fish (Paralichthys olivaceus) (Jung, K. S., 2002), however its affect on fish immune function and oral delivery of bioagent has not been studied. Similarly, in vitro binding of SBA to bovine small intestinal brush-border membranes has been reported (Hendricks, et al., 1987), however its affect on bovine immune function and oral delivery of bioagents has not been contemplated until herein.

Respiratory Bioactivity. SBA binds to the dorsal epithelium branchial arches and hatching gland cells (HGCs) of brown trout and to the olfactory mucosa cells in brachiopterygian fish (Polypterus, Erpetoichthys). SBA stains type I alveolar cells in mini-pigs. SBA activity on immune cells in the respiratory system has not been studied.

Endocrine Bioactivity. Cholecystokinin (CCK) has been classically defined as a duodenal cell-derived gastric peptide hormone responsible for stimulating fat and protein digestion via pancreatic secretion of digestive enzymes (e.g, trypsinogen, chymotrypsinogen, amylase, lipase). CCK has been shown to play a role in intestinal neuroimmuoregulation. For example, CCK stimulation of monocytes induces production of inflammatory cytokines such as TNF, IL-1, IL-6 and IL-8. CCK expressing enteroendocrine cells are also stimulated by cytokines IL-4 and IL-13 produced from intestinal CD4+ T cells. High levels of CCK-8 have been shown to inhibit T cell function through inhibition of cell mobility and mitogen-induced proliferation. A dose-dependent increase in dietary SBA results in increased plasma CCK levels and stimulates CCK release from rabbit jejunal cells. SBA induces secretion of alpha-amylase from rat pancreatic acini in vitro a result likely caused by SBA induction of CCK release. Modulation of oral delivery of bioactive agents through the endocrine-immune system axis using soybean-derived lectins has not be studied.

Pathogen interactions. The direct interaction of SBA with viral type I and type II glycoproteins has not been widely studied. SBA was reported to bind to herpes simplex virus type I-specific gC glycoprotein by affinity chromatography. SBA was also shown to enhance Hantaan virus infection in vitro, presumably through functioning as a cross-linking ‘bridge’ between the viral envelope and the cell surface. The use of receptor specificities associated with lectins and other plant-derived ligands can have a number of different effects on the activities of the biological molecules or agents that are targeted. The effects of such ligand-specific binding interactions on, for example, the immunological properties of vaccines directed against specific pathogens has not previously been characterized.

Immune Effects. Increasing doses of purified SBA in rat diets had a negative effect on both cell-mediated cytokine production and humoral immune function (Tang, S., 2006). Although porcine plasma lymphocytes bind SBA (Sage, H. J., 1982), the immunomodulatory effect of SBA on porcine immune cells is also not known. The in vitro or in vivo effects of SBA (nor ranges in concentration and effects has been tested) on the piscine, avian, bovine, feline, canine and equine immune systems have not been reported in the scientific literature. The monomeric or multimeric forms of the lectins for these biological activities is not known, and most certainly is affected by pH, concentration, bio-enhancement/neutralization by other biomolecules in a mixture or in the gut.

Red Kidney Bean (Phaseolus vulgarus) Lectin (PHA)

Biochemistry. PHA is a homotetrameric (115-120 kD) protein lectin comprised of five isolectins (L4, L3E1, L2E2, L1E3, and E4), based on their relative lymphocyte- and erythrocyte-reactive biologic activities (Leavitt, R. D., 1977). All forms show a 33 kD subunit band by SDS-PAGE. PHA exhibits extreme pH stability, especially in the acidic range, existing as a dimer at pH 2.5 and as a tetramer at pH 7.2. PHA is also thermostable with no loss of bioactivity following heat treament at 70° C. for up to 4 hours and retention of partial activity after 3 hours at 90° C. However, beans presoaked overnight before cooking lost all activity after 10 min at 100° C. E(4) and L(4) isoforms have high specificity for complex type N-glycans containing bisecting GlcNAc or a beta 1,6-linked branch, respectively. Like SBA, the effect of varying concentration and/or physical environment of PHA, and of combination with other active components, on the physiological and/or immunological activities induced by PHA has not been examined. The effects of PHA-like lectins from fish meal or krill on target animal immunology and physiology have also not been well characterized.

Intestinal Bioactivity. PHA binds to the gut epithelium of suckling rats, causing villi shortening, increase in crypt cell proliferation, hypertrophy, and functional maturation of the GI tract. PHA isolectins E4 and L4 can bind in vitro to swine jejunal enterocytes and decrease the villus:crypt height ratio. Exposure of piglets to a crude red kidney bean lectin for 3 days prior to weaning induced positive changes in both performance and small intestinal functional properties. PHA-E and -L failed to stain Peyer's patches immune cells in the porcine ileum. The dietary intestinal effects of PHA in piscine, avian, and other mammalian species has not been reported. Dietary PHA can decrease levels of heat shock proteins (HSPs) in rat gut epithelial cells and enterocyte-like Caco-2 cells. These decreased levels of stress proteins may leave these cells more susceptible to the harmful content of the gut lumen.

Respiratory Bioactivity. PHA staining of alveolar and other cells in the respiratory tract of piscine, avian, porcine, bovine and other mammalian species has not been reported.

Endocrine Bioactivity. Similar to SBA, PHA is a well-known activator of enterocyte CCK secretion. In addition, PHA induces dose-dependent secretion of trypsinogen, chymotrypsinogen, and alpha-amylase by the pancreas in vivo.

Pathogen interactions. PHA binding to viral glycoproteins has not been widely reported, presumably due to the lack of specific sugar specificity. PHA is known to bind Ebola virus glycoprotein. PHA reportedly binds to proteins from Myxobolus cerebralis, the parasitic causative agent of salmoid whirling disease. As discussed above, the effect of binding-specific interactions on immunogenicity or other physiological effects of bioagent delivery has not been previously characterized in the context of lectin or other ligand content for vaccines and other compositions. Depending on the physical context, the type of lectin or other ligand, the target pathogen and subject animal, binding interactions may enhance, diminish or otherwise modify the immunological or physiological effect of the bioactive agent.

Immune Effects. PHA is a well-known mitogen for piscine, avian and mammalian T lymphocytes. For examples, in vitro treatment of fish T leukocytes with PHA causes secretion of IFN-gamma (Zou, J., 2005). Mice orally fed purified PHA can prevent oral tolerance against a surrogate food protein and induces a plasma PHA-specific antibody response (Kjaer, T. M., 2002).

Lima Bean (Phaseolus lunatus) Lectin (LBL)

Biochemistry. LBL was the first plant haemagglutin shown to possess human blood group specificity. Agglutinating activity is present in two isolectins with relative molecular weights of 110-138 kDa and 195-269 kDa. It has been suggested that LBL isolectins exist in both tetramer and dimer forms comprised of glycosylated subunits of approximately 31 kDa. The two isoforms, designated LBL1 and LBL2 are encoded by a multigene family. LBL shows specificity for the trisaccharide, GalNAc alpha 1-3[Fuc alpha 1-2]Gal beta 1-R and N-acetyl-D-galactosamine is a specific and reversible inhibitor of both isoforms. As with the other lectins, concentration, physical environment and interaction with other components of the composition may act to modify the effects obtained with oral delivery of bioactive agents.

Intestinal Bioactivity. In contrast to SBL and PHA, the intestinal bioactivity of LBL has not been extensively studied. One report showed that dietary sub-lethal doses of LBL in rat had marginal effects on organ weights, and pancreatic and intestinal trypsin and chymotrypsin activities.

Respiratory Bioactivity. LBL staining of alveolar and other cells in the respiratory tract of piscine, avian, porcine, bovine and other mammalian species has not been reported

Endocrine Bioactivity. LBL stimulation of enterocyte CCK secretion has not been reported.

Pathogen interactions. LBL interaction with glycoconjugates from viruses or parasites has not been reported.

Immune Effects. The octomeric form of LBL is a known mitogen for human lymphocytes (Ruddon, R. W., 1974), whereas the tetrameric form is not, unless it is first chemically cross-linked. Neither form is reportedly mitogenic for bovine lymphocytes.

### Isoflavones

Although the present discussion is focussed on isoflavones, the skilled artisan will realize that other types of flavonoids or flavonoid derivatives may also be of use in the claimed compositions and methods. For example, the flavonoid quercetin has been reported to have anti-inflammatory activity by inhibiting the initial process of inflammation and may also affect immunogenicity or other properties of orally delivered bioactive agents. Epicatechin, oligomeric proanthocyanidins, hesperidin, rutin, luteolin, apigenin, myricetin, naringenin, tangeritin, catechins and other flavenoids may be found in a variety of plant materials, such as citrus fruit, berries, onions, parsley, legumes, green tea and dark chocolate.

The early intermediates (genistein, diadzein, and biochanin A) of the isoflavone metabolic pathway are phytoalexins typical of the Fabaceae. These dietary isoflavones have been shown to display a wide range of weak estrogen receptor ligand effects. The most common dietary source of isoflavones is soybean, followed by faba beans and peanuts, although chick pea and alfalfa also contain isoflavones. The 3 main isoflavones show high affinity binding to rainbow trout liver estrogen receptors (genistein>daidzein>biochanin A). Isoflavones have been shown to inhibit angiogenesis in chick chorioallantoic membranes. The activity of isoflavones especially from ground powders of native source material on immune cells from piscine, avian and several other mammalian species has not been reported. Nor have the effects of concentration, physical environment or combination with other components on immunological or physiological activities been characterized.

### Genistein

Genistein (4′5,7-trihydroxyisoflavone) occurs as a glycoside (genistin) in the plant family Leguminosae, which includes the soybean (Glycine max). Genistein can regulate immune function (Cooke, P. S., 2006) and some evidence in mice suggests that it can suppress cell-mediated immune responses. Treatment of murine splenocytes with genistein at concentrations from 10⁻⁶ and 10⁻⁷ M significantly decreased the IFNgamma/IL-10 ratio, suggesting a shift in the Th1/Th2 balance towards a Th2 response (Rachon, D., 2006). Genistein can enhance IL-4 production in activated murine T cells. Genistein has suppressive effects in macrophages by inhibiting nitric oxide (NO) production through the inducible nitric oxide synthase (iNOS) enzyme (Choi, C., 2003).

### Daidzein

Dietary daidzein additions resulted in improvements in daily pig weight gain, daily feed intake, and gain/feed during periods of peak viremia (d 4 to 16 after inoculation) following PRRSV challenge. In addition, soy daidzein at dietary concentrations of 200 to 400 ppm is an orally active immune modulator that enhances systemic serum PRRSV elimination and body growth in virally challenged pigs. At high dietary doses (20, 40 mg/kg), daidzein is stimulatory to the three main arms of the immune system: innate, cell-mediated and humoral immune response (Zhang, R., 1997). Daidzein can potentiate the effect of mitogens on murine lymphocyte activation and secretion of IL-2.

### Biochanin A

The estrogenic activity of biochanin A (5,7-dihydroxy 4′-methoxy isoflavone) is several orders of magnitude lower when compared to other structurally related isoflavones. However biochanin A can be converted to genistein by intestinal microflora and by liver cytochrome 450 (CYP450) isoenzymes. As discussed above, the interaction between probiotic agents and bioavailability of orally delivered bioactive agents, for example due to enzymatic conversion of compounds, has not been previously investigated. Biochanin A can enhance IL-4 production in activated murine T cells and reduces hydroxen peroxide induced production of inflammatory cytokines (TNF, IL-6) and nitrite oxide in osteoblasts.

### Fatty Acids

Soybeans contain both n-3 and n-6 polyunsaturated fatty acids (PUFA) with higher potency in the n-3 form. These compounds have innate anti-inflammatory properties and block eicosanoid production (e.g., prostaglandins). These fatty acids are also immunosuppressive by decreasing the availability of T cell receptors required for activation and inhibiting production of Th1 cytokines (Zhang, P., 2005). PUFA are also able to influence the amount of LPS-induced NO generated through the iNOS enzyme in murine macrophages. The effect of soybean derived PUFAs on the piscine immune system has not been reported. In chickens, immune organ growth (thymus, spleen, bursa) was impacted significantly by the amount of dietary PUFA and the ratio of n-6 to n-3 fatty acids. In rats, the dietary ratio of n-6/n-3 PUFA is important for the induction of neonatal oral tolerance.

As with the other components of the claimed compositions, the effects of concentration, binding specificity, physical environment (pH, etc.), and combination with other active agents such as isoflavones and lectins on the immunological or physiological effects of polyunsaturated fatty acids, alone or in combination with orally delivered bioactive agents, has not previously been determined. The activity of fatty acids especially from ground powders of native source material on immune cells from piscine, avian and several other mammalian species has not been reported.

### Soyasaponins

These compounds are found almost exclusively in soybean and kidney beans and can function as a vaccine adjuvant by enhancing immune responses to ovalbumin in mice. There are several different types, including soyasaponin A(1), A(2), and I, group B, deacetylated and acetylated forms, soyasaponin III and soyasapogenol B monoglucuronide. Soyasaponin A(1) has a long sugar side chain and induces stronger total antibody responses than soyasaponin A(2). In general the adjuvant potency of these compounds is soyasaponin I>soyasaponin II>soyasaponin III. These compounds contain sugar side chains and may interact with bean lectins. Soyasaponins are not degraded during gut passage in Atlantic salmon (Salmo salar L.) (Knudsen, D., 2006). The effect of soyasaponins on piscine and some mammalian species has not been reported. Interactions of soybean lectin, soyasaponins, and glycinin with rabbit jejunal mucosa in vitro suggest that soybean lectin binding to terminal galactoside sites at the enterocyte apical membrane, enhances a crenator effect of saponins that leads to increase leakage of glycinin into intestinal cells. Saponins from unique sources such as beans, in finely ground formulations, for oral delivery, antigen stability, immune/physiological stimulation, and oral delivery in various target species have not previously been described.

Saponins may have immune modulatory effects. Saponins derived from Pleurospermum kamtschaticum have been reported to have inhibitory effects on NO, prostaglandin E₂ (PGE₂), and tumor necrosis fator-alpha (TNF-alpha) in murine macrophages (Jung, H. J., 2005). In addition, the saponin fraction from Gleditsia sinensis has been reported to inhibit LPS-induced NO and interleukin-1-beta (IL-1-beta) production in murine macrophages.

As with the other components of the claimed compositions, the effects of concentration, binding specificity, physical environment (pH, etc.), and combination with other active agents such as isoflavones and lectins on the immunological or physiological effects of saponins, alone or in combination with orally delivered bioactive agents, has not previously been determined.

### EXAMPLES

The following Examples are included to demonstrate preferred embodiments of the invention. It should be appreciated by those of skill in the art that the techniques disclosed in the Examples which follow represent techniques discovered to function well in the practice of the invention, and thus can be considered to constitute preferred modes for its practice. However, those of skill in the art should, in light of the present disclosure, will appreciate that many changes can be made in the specific embodiments which are disclosed and still obtain a like or similar result without departing from the spirit and scope of the invention.

### Example 1

### Compositions for Bean-Specific Lectin Targeting of Vaccine Antigens

Compositions comprising lectins (Table 1) are advantageous for the delivery of bioactive agents, such as vaccine targeted antigen delivery through direct binding of lectin to enterocytes and intestinal antigen presenting cells (e.g, epithelial cells and M cells). Bean-specific lectins may specifically bind vaccine antigens and specifically target their delivery to distinct regions of the intestinal system that exhibit enterocyte expression of the appropriate carbohydrate ligand(s) specific for the lectin (Table 1). Studies are conducted to assess and map the intestinal binding location(s) of bean-derived extracts or purified lectins. Compositions are specifically designed to improve vaccine antigen delivery to immune cell rich areas of the gastrointestinal tract. Compositions for vaccine antigen delivery are designed for the fish or animal species of interest for vaccine delivery.

As discussed above, either in vitro cell-based assays or in vivo animal models may be used to assess the effects of different formulations on oral delivery of bioactive agents. Various model systems are known in the art for measuring the efficacy, dosage, uptake, delivery and/or immunological or physiological effects of bioactive agents and any such known model may be used.

TABLE 1
Mitogenic Lectins (Leguminosae)

| | | | MW | |
|---|---|---|---|---|
| Species | Common Name | Lectin | (kDa) | Sugar Specificity |
| Glycine max | soybean | SBA² | 110 | GalNac¹, galactose |
| Phaseolus | lima bean | PHA-L | 126 | complex |
| limanis (lunatus) | | | | oligosaccharides, |
| | | | | GalNac¹ |
| Phaseolus | white or red | PHA-E | 128 | complex |
| vulgaris | kidney bean | | | oligosaccharides |
| Vicia faba | fava bean, | LBL | 50 | Mannose, glucose |
| | broad bean | | | |
| Vicia sativa | Vetch | VSA | 70 | Mannose, glucose |
| Pisum sativum | small pea | PSA | 49 | Mannose, glucose |
| Lens culinaris | lentil | LCA | 49 | Mannose, glucose |
| Lathyrus | sweet pea | LOA | 53 | Mannose, glucose |
| odoratus | | | | |
| Canavalia | jack bean | Con A | 102 | Mannose, glucose |
| ensiformis | | | | |
| Abrus | Rosary pea, | APA | 134 | galactose |
| precatorius | jequirity bean | | | |
| Arachis | peanut | PNA | 120 | GalNac¹ |
| hypogaea | | | | Galactose |

¹N-acetyl-D-galactosamine
²mitogenic for neuraminidase treated lymphocytes

To evaluate the effects of lectins from bean extracts on biological activity of bioagents used for oral delivery two exemplary formulations were prepared. Formula 1 (Aviare) contained the following composition of dry ingredients by weight: soyabeans (20%), lima beans (15%), deoxycholate (0.5%), dried ovalbumin (10%), calcium carbonate (10%), EDTA (5%), sodium bicarbonate (2.5%) and standard grower poultry feed (37%). Formula 2 (A-03) contained soyabeans, white beans, dark red kidney beans and lima beans (10.47% each), bentonite (3.14%), deoxycholate (0.52%), Brewer's yeast (5.24%), Betafin S4 (2.09%), fish meal (15.71%), dried ovalbumin (5.24%), calcium carbonate (10.47%), EDTA (5.24%), sodium bicarbonate (10.47%) and soy oil (10% of total mixture). Dry ingredients were combined together and then milled through a 0.6 mm mesh, using a hammermill (Bliss, Ponca City, Okla.) and stored at room temperature. The resulting powders were then used for spiking and recovery studies using Newcastle disease virus (NDV). The formulations for F3, F4, F5 and F6 compositions discussed below are the same as F2, with the addition of further bean powders.

For certain studies, four additional exemplary formulations were prepared. Formula 3 contained an additional 1.0 g soybeans, Formula 4 contained an additional 1.0 g lima beans, Formula 5 contained an additional 1.0 g white kidney beans and Formula 6 contained an additional 1.0 g dark red kidney beans. The formulas were then used for additional spiking and recovery studies using Newcastle disease virus (NDV).

Formula 7 (F7) contained soyabeans (10%), bentonite (3%), deoxycholate (0.5%), Betafin S4 (2%), fish meal (9.5%), dried ovalbumin (30%), calcium carbonate (10%), EDTA (5%), sodium bicarbonate (5%), krill meal (15%) and krill oil (10% of total mixture).

Newcastle disease virus (NDV) is a well characterized antigen for use in vaccine development. The antigens are well defined and the immunological response and disease model are codified in Code of Federal Regulations 9 (9 CFR) part 113. The Lasota strain of NDV was prepared as inactivated virus preparations from allantoic fluid of infected embryonated eggs. Pre-inactivation titers of NDV were 10⁸ to 10⁹ EID₅₀ /ml. Inactivation of the virus preparations was performed using binary ethylene amine (BEI). The inactivated virus preparations were checked for viable virus using 10-day old embryonated eggs inoculated as described in the Code of Federal Regulations Title 9, Chapter 113.37 (9 CFR 113.37). Protein quantification of biological preparations was determined using a BCA assay kit as described by the manufacturer (Pierce Chemical Co).

Sodium dodecyl sulfate polyacrylamide gel electrophoresis (SDS-PAGE) and Western Blots. Sodium dodecyl sulfate polyacrylamide gel electrophoresis (SDS-PAGE) was performed using Novex, Western Breeze and NuPAGE systems as described by the manufactuer Invitrogen (Carlsbad, Calif.). Samples for treatment were diluted to appropriate levels in TEN buffer and then mixed 1:1 with reducing buffer. Samples in reducing buffer were then incubated 10-15 minutes at 70° C. using an Eppendorf thermomixer (constant mixing at 1400 rps). To prepare electrophoresis running buffers, 20× MES (2-[morpholino] ethanesulfonic acid) was diluted 1:20 in 1 L of distilled water from a 20× stock (prepared by mixing 10 g MES powder per liter of distilled water, adjusted to pH 5.7 using 1 N sodium hydroxide). Running buffer (200 ml) was removed and 500 ul of NuPAGE antioxidant added. Gels were NuPAGE 12% Bis-Tris gels. A power supply (ECL 458) was attached and the gels were run at constant voltage with the positive (cathode) pole at the bottom of the gel (200 V for 35 minutes).

Staining and Western blot of SDS-PAGE. NuPAGE gels were removed from the cassettes and placed in a shallow plastic tray, and 100 mls of Coomassie blue stain was added. After 30 minutes incubation, the gel was destained in 5% methanol (v/v) and 7.5% acetic acid (v/v) in distilled water for 1 hour. The gel was rinsed with fresh destaining solution and destained overnight. The gel was removed from the destain solution, rinsed with distilled water and dried under vacuum overnight onto immobilized porous cellophane. For Western blots, transfer buffer (10% methanol, 1× NuPAGE transfer buffer and 0.1% NuPAGE antioxidant) was prepared and nitrocellulose membranes (Novex) and sponge pads (Novex) were incubated in the buffer 10-20 minutes before use. The wet gel was then placed onto the nitrocellulose, and sponge pads were placed on either side. The entire gel and pads were placed into an Xcell Blot module (Novex) and placed in the Xcell blot apparatus. The outer wells were filled with transfer buffer, a constant voltage supply was attached and the transfer was performed at 30V for 60 minutes. The membrane and gel were removed from the module and the membrane was placed in a shallow plastic tray. Ten ml of primary antibody diluted in blocking solution was then added to the tray, and incubated with the blot at room temperature for 1 hour. The membrane was rinsed in blocking buffer and then 10 ml of conjugated secondary antibody in blocking buffer was added and incubated an additional hour at room temperature. Blocking buffer, incubation buffers and detection buffers were all supplied and utilized according to Novex Western Breeze™ Immunodetection System.

Quantitative HN ELISA. ELISA plates were coated with Chicken anti-NDV polyclonal antibody (SPAFAS Lot no. CO116) (1/2000 final dilution in 10 mM borate buffer, pH 9.0) and incubated overnight at 4° C. Plates were equilibrated to R.T., washed 3× with PBS-Tween (PBS-T), incubated for 2 hr at 37° C. in blocking buffer (5% w/v skim milk in PBS-T) and washed 1× in PBS-T. Semi-purified HN antigen derived from inactivated NDV strain LaSota was used in all assays as a standard reference control. Plates were incubated for 1 h at 37° C. and then washed 3× in PBS-T. A 1:4000 dilution of mouse anti-NDV-HN specific monoclonal 4A (Dr. Iorio, Dept. of Molecular Genetics and Microbiology, Univ. of Mass. Medical School) was added and plates were incubated for 1 h at 37° C., washed 3× in PBS-T, and incubated for 1 h at 37° C. with a 1:6000 dilution of anti-mouse IgG (H+L) HRP (KPL, Inc., Gaithersburg, Md.). Plates were washed 3× in PBS-T and ABTS substrate (KPL, Inc.) added. The reaction was monitored using a kinetic plate reader (Tecan Sunrise, Tecan, Research Triangle Park, N.C.), and read when the OD_{405/490} nm of the initial dilution of standard reference antigen was between 0.7-1.0. Data was transported for linear regression and quantitation analysis (Microsoft Excel 2000 version 9.0.3821 SR-1).

Hemagglutination Assay. A chicken or rabbit red blood cell (RBC) hemagglutination endpoint assay (HA) was used to determine the HA titer for each treated sample. Briefly, serial two-fold dilutions of each sample (triplicate) were prepared in 96-well round bottom plates. Freshly prepared 1% chicken or rabbit RBC solution was added to each well and plates placed on a plate shaker (600 rpm, 20-30 sec). Following incubation for 1 hr at 5° C., plates were read for agglutination. HA endpoint titers were calculated based on the reciprocal of the highest dilution to exhibit 100% agglutination.

Spike and Recovery test using Formula 1 or 2. Freeze dried and inactivated NDV antigen derived from allantoic fluid was rehydrated in Dulbecco's Phosphate Buffered Saline (DPBS) and mixed with Formula 1 or Formula 2 material in the following ratios. The oral delivery formula (0.6 g) was mixed with 0.4 ml of buffer or oil followed by 1.2 ml of the inactivated and rehydrated NDV. The entire mixture was incubated at room temperature for 30 minutes followed by centrifugation to remove the solids, the supernatant was decanted and immediately tested by HN ELISA and Western Blot.

TABLE 2
ELISA Results of Killed NDV Spike and Recovery
in Oral Delivery Compositions

| | | Recovered |
|---|---|---|
| | Spiked HN Concentration | HN Concentration |
| NDV Sample | (μg HN/mL) | (μg HN/mL) |
| NDV AF | Not applicable | 3.1 |
| (post-lyophilization) | | |
| NDV AF + F1 carrier | 3.1 | 2.3 |
| feed | | |
| NDV AF + F1 carrier | 3.1 | 3.3 |
| feed with Oil | | |
| NDV AF + F2 carrier | 3.1 | 1.0 |
| feed | | |
| NDV AF + F2 carrier | 3.1 | 1.5 |
| feed with Oil | | |
| NDV AF + Buffer | 1.55 | 1.5 |
| NDV AF + Buffer with | 1.55 | 1.6 |
| oil | | |

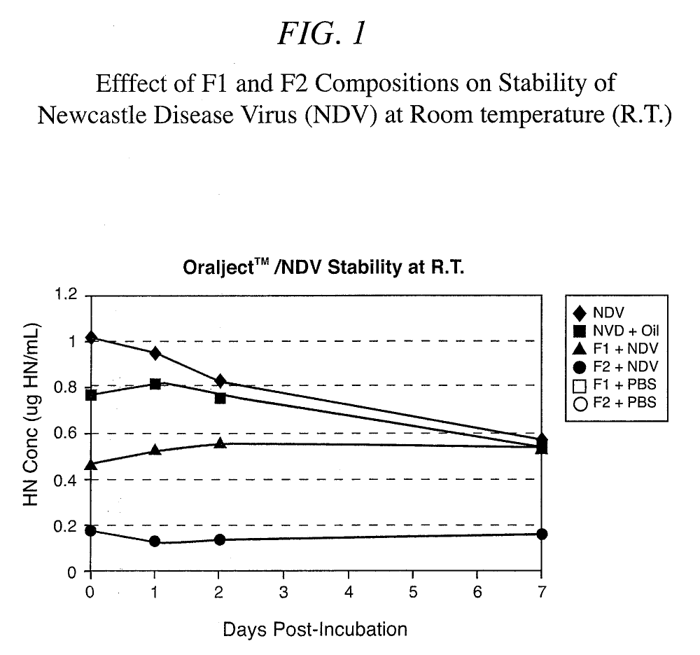

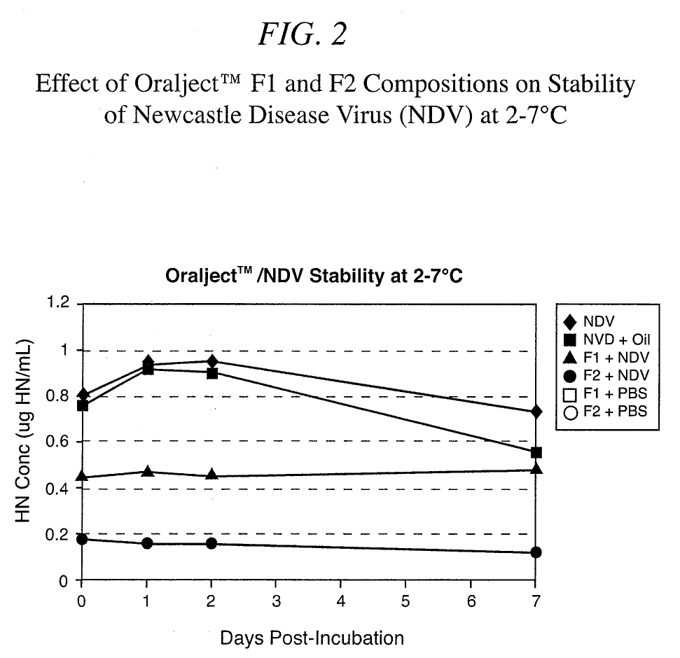

The results in Table 2 indicate that the recovery of ELISA signal for NDV-HN protein could readily be detected from F1 composition, but the signal was significantly reduced by F2 composition. When evaluated over time the the signal of the NDV-HN ELISA at room temperature (FIG. 1) and at 2-7° C. (FIG. 2) are presented.

At room temperature (FIG. 1) the HN ELISA signal slowly decreased, which is typical of this antigen. It is sensitive to temperature, detergents, drying agents, and light, which cause it to decay without stabilization compounds or by cryopreservation. Combining the antigen with F1 reduced the signal by approximately 56% but the signal increased rather than decreased over the next few days, indicating that the HN was equilibrating in the F1 composition. In contrast, in F2 the signal was reduced by 80% immediately after mixing, however, the signal was remarkably stable in both formulations, indicating that the HN antigen can be stabilized using the disclosed compositions. Similar effects were observed at 2-7° C. (FIG. 2).

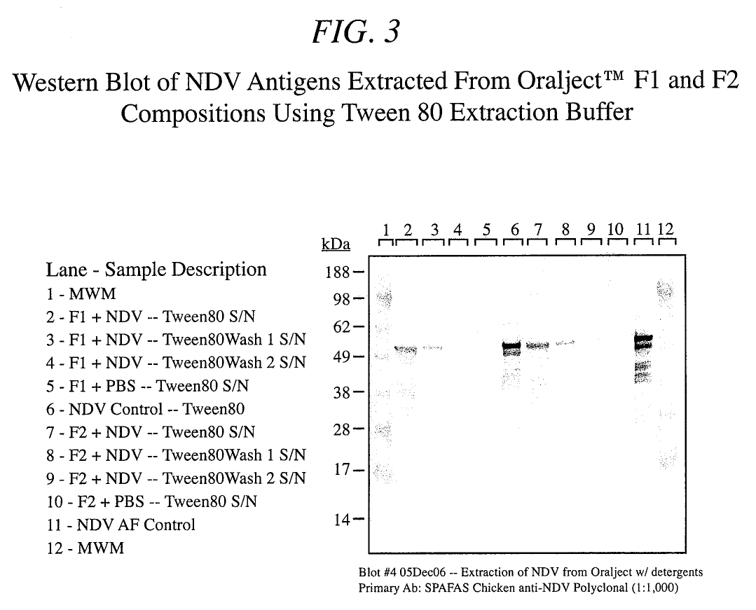

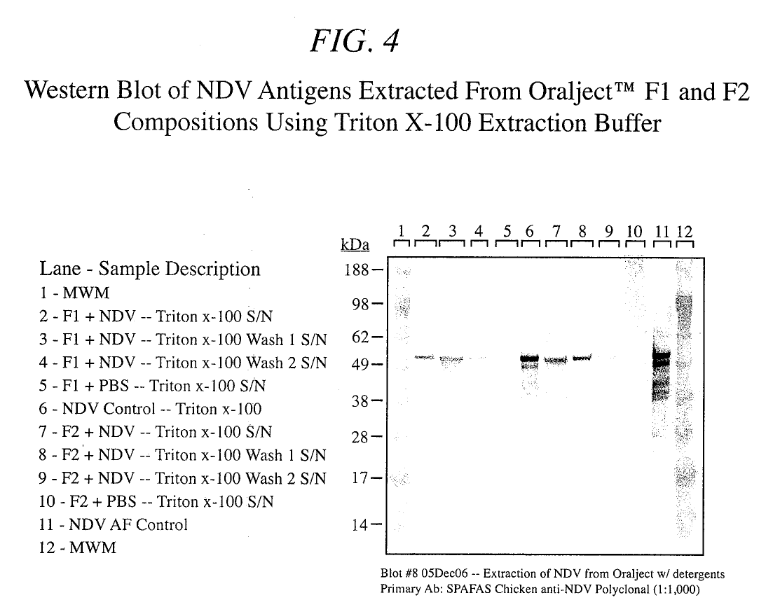

When evaluated by SDS-PAGE and Western blot the data showed that both formulations provided a stabilization of NDV proteins that could be recovered intact. However, the recovery from F1 can easily be affected using a mild detergent Tween 80 (FIG. 3), whereas the recovery of HN from F2 was not completely recovered even with a more harsh detergent such as Triton 100 (FIG. 4). Extraction from F2 compositions revealed a profile more similar to the non-treated controls (Compare lanes 6 and 11 in FIG. 3 and FIG. 4), than F1 compositions, indicating a differential binding of NDV with F1 compositions from F2 compositions. The data provide evidence that components within each formulation provide different biological binding while providing a stable environment for a protein that is typically volatile to most ambient conditions.

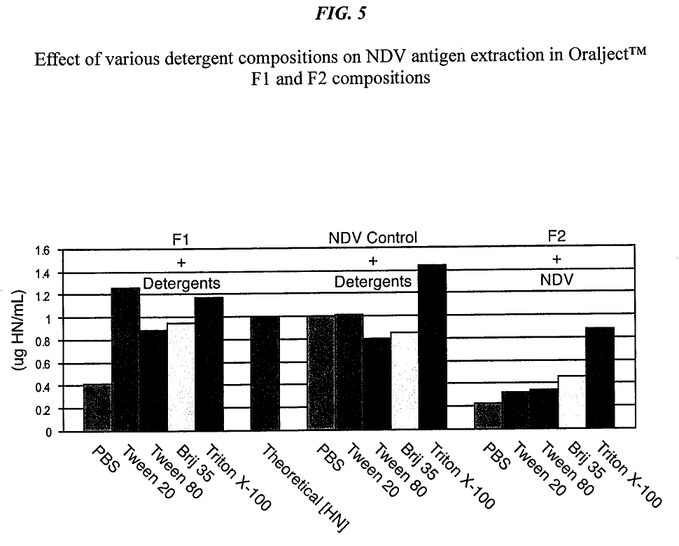

The results shown in FIG. 5 illustrate recovery of ELISA signals for NDV protein using various detergents in F1 and F2 compositions. The signal was reduced when mixed in buffer in both compositions, however, in F1 the signal was recovered in a manner similar to the NDV control, whereas in F2 compositions the signal was not recovered; only Triton 100 provided recovery of signal to similar levels as NDV controls. The reduction of signal after addition of either composition is indicative of binding or blocking of signal. Since the signal cannot be recovered even after detergent extraction, this indicates a binding rather than signal blocking.

The results suggest that Triton 100 allows for free exchange of the substrate between receptors on the virus and lectins of the bean extracts that impair signal due to the inability of substrate saturated virus particles to be caputured in the HN-specific ELISA. The results showed that the lectin components of the compositions can be used to stabilize vaccine antigens in compositions of finely ground bean extracts or powders. The concentration of these lectins, ratios of lectins, and biological function of lectins can be used to accommodate a large selection of biological agents as described above. The composition of bean extracts or other biological sources for similar components of the compositions can be utilized to not only stabilize bioactive components but provide selective stimulation of specific immune (e.g., innate, T helper type 1, T helper type 2, T cytotoxic, B-cell) or physiological functions in the target animal, based on the ratio of different extracts and consequent concentrations of lectins, isoflavones, fatty acids and soyasaponins which occur naturally in extracts of beans.

Spike and Recovery test using Formulas 3-6. Freeze dried and inactivated NDV antigen derived from allantoic fluid was rehydrated in Dulbecco's Phosphate Buffered Saline (DPBS) and mixed with ground bean formulas 3-6 in the following ratios. The oral delivery ground bean formula (1.0 g) was mixed with 1.2 ml of the inactivated and rehydrated NDV. The entire mixture was mixed by vortexing and incubated at room temperature for 10 minutes. Then 2.5 mls of PBS alone or PBS+0.5% Triton X-100 (extraction buffer) was added, mixed for 10 minutes followed by centrifugation (2900×g, 10 minutes, 4° C.) to remove the solids, the supernatant was decanted and immediately tested by HN ELISA.

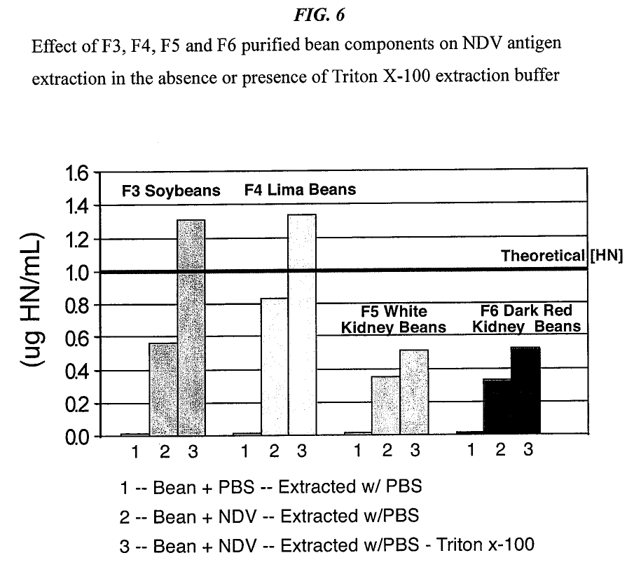

The results shown in FIG. 6 illustrate recovery of ELISA signals for NDV protein using detergent in ground bean formulas 3 (F3), 4 (F4), 5 (F5), and 6 (F6) compositions. The signal is reduced when mixed in buffer containing each of the four ground bean compositions. The reduction of signal after addition of each individual bean extract composition is indicative of binding or blocking of signal. Importantly, the addition of Trixton X-100 to F3 (soybean) and F4 (lima bean) compositions resulted in complete recovery of signal. In contrast, the addition of Triton X-100 to F5 (white kidney bean) or F6 (dark red kidney bean) compositions failed to recover signal. Since the signal cannot be recovered even after detergent extraction, this indicates a binding rather than signal blocking. These data indicate that the presence or absence of specific lectin-containing bean components can influence the ability to specifically bind bioagents used for oral delivery.

### Example 2

### Effect of Different Formulations on In Vitro Immunological Activity

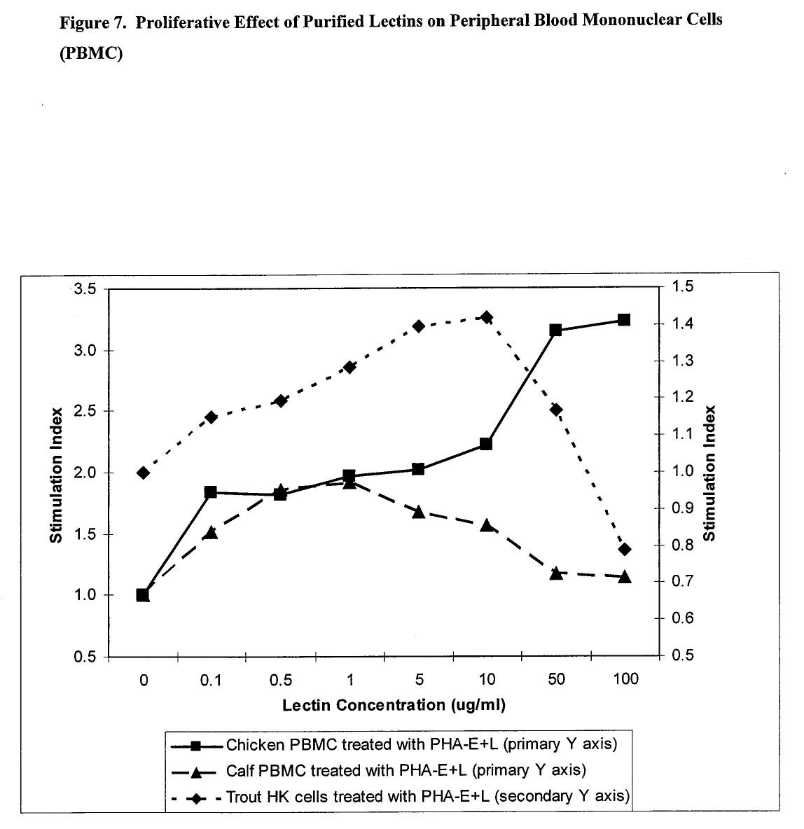

Compositions for bean-specific lectin activation of immune cells. Compositions containing mitogenic lectins (Table 1) are advantageous for vaccine stimulation of intestinal immune cells (e.g., T cells and B cells) and enteroendocrine cells secreting the immunoendocrine peptide, CCK. CCK secretion causes upregulation of proinflammatory cytokines (TNF, IL-1, IL-8) and activation of the innate immune response. Tables 3-7 and FIG. 7 provide exemplary types of lectin components that may be of use to achieve different desired effects of immune or endocrine stimulation. As disclosed in Table 1, such components are present in different concentrations and occur in different naturally occurring sources. The claimed compositions are formulated to optimize lectin content for immunologic effects directed to different pathogens and in different recipient host species. Studies are conducted to assess the CCK secretion potential of different bean-derived extracts using a CCK-enteroendocrine cell line (Example 4). Compositions comprised of specific combinations of bean-derived extracts and finely ground powders are formulated to improve CCK release by enteroendocrine cells.

Compositions for bean-specific lectin binding to vaccine antigen and increased antigen stability. Compositions containing lectins (Table 1) are advantageous for vaccine antigen stability. Bean-specific lectins bind vaccine antigens that express the appropriate carbohydrate ligand(s) specific for the lectin (Table 1). Studies are conducted to assess the effect of different lectin source materials (Table 1) and mass to surface ratio of finely ground bean extracts and powders on antigen stability, as described in Example 1 above. The sources and amounts of natural ingredients in the claimed compositions are optimized to enhance stability of specific pathogenic antigens in different target species.

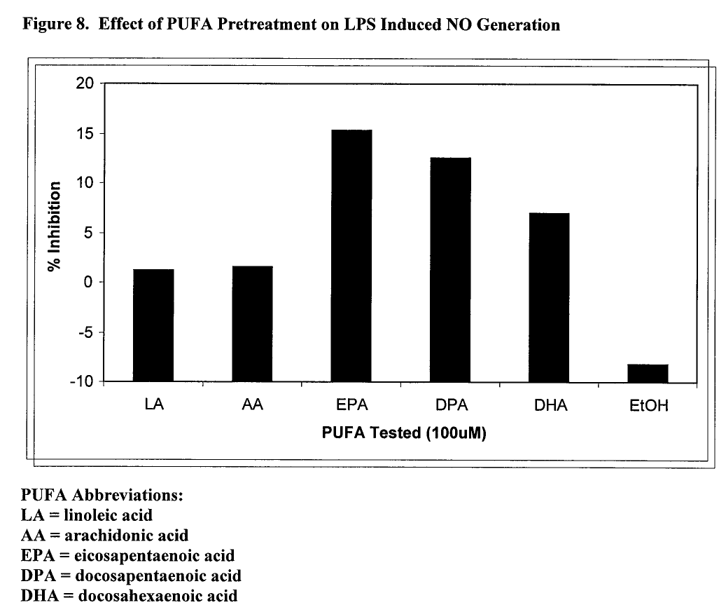

Compositions for fatty acid activation of immune cells. Compositions containing n-3 and n6-polyunsaturated fatty acids are advantageous for vaccine stimulation of intestinal immune cells (e.g., T cells and B cells). They are also of use to control the effect of oral tolerance to vaccine antigens in the targeted animal species. Table 8 and FIG. 8 disclose exemplary naturally occurring compounds of beans of use for immunostimulatory activity of bean-derived n-3 or n-6 polyunsaturated fatty acids against a panel of immune cells from different target species of interest. Different concentrations of bean-derived, n-3 or n-6 polyunsaturated fatty acids are obtained by varying the source and amount of plant-derived materials, and mass to surface ratio of finely ground bean extracts and powders incorporated into the final compositions to determine optimal compositions for vaccine formulations. Prototypic cell types representative of target animal cells may be used to obtain data in exemplary species.

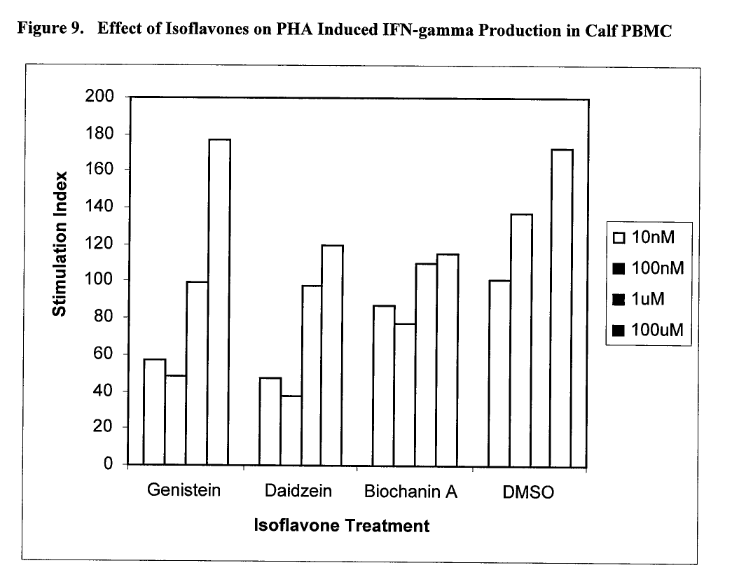

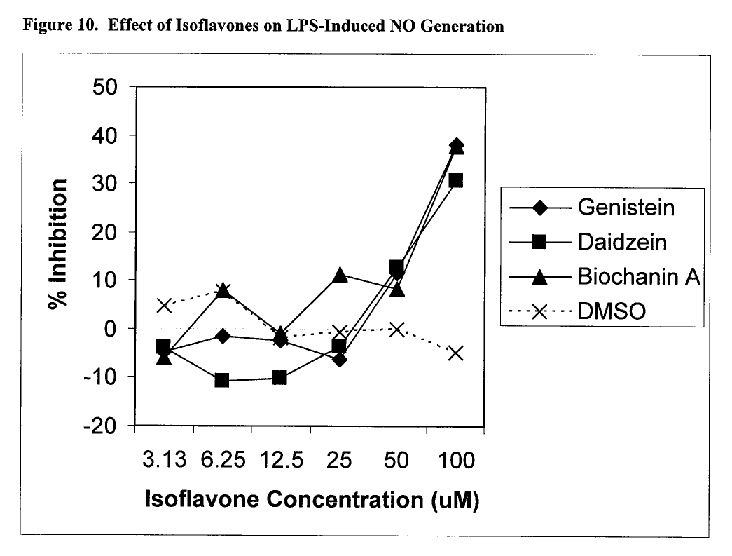

Compositions for isoflavone activation of immune cells. Compositions containing isoflavones are advantageous for vaccine stimulation of intestinal immune cells (e.g., T cells and B cells). Studies are conducted to assess the immunostimulatory activity of bean-derived isoflavone-containing extracts against a panel of immune cells isolated from different species of interest for oral vaccine delivery (Example 6, Table 9, FIGS. 9 and 10). Different concentrations and sources of bean-derived extracts, and mass to surface ratio of finely ground bean extracts and powders are tested to determine optimal formulations for different vaccines. Compositions comprised of specific bean-derived isoflavones can be used to improve vaccine activation of immune cells in the gastrointestinal tract. Compositions for isoflavone activation of immune cells are designed for the fish or animal target species of interest.

Compositions containing bean-derived soyasaponins as vaccine adjuvants.

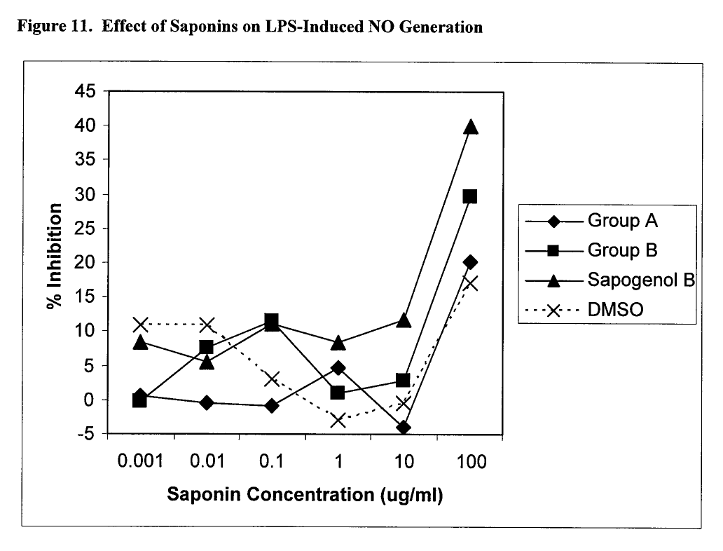

Compositions containing bean-derived soyasaponin vaccine adjuvants are advantageous for vaccine stimulation of antigen-specific antibody responses. Studies are conducted to assess the immunostimulatory activity of bean-derived extracts containing soyasaponins (Example 7, FIG. 11). Different sources and amounts of plant materials containing soyasaponins and mass to surface ratio of finely ground bean extracts and powders are tested to determine optimal compositions for vaccine formulations. Compositions containing specific plant-derived soyasaponins are of use as vaccine adjuvants to improve antigen-specific activation of immune cells in the gastrointestinal tract. Compositions comprising plant homogenates containing specific soyasaponins are optimized for the fish or animal target species of interest.

### Methods and Materials

Bean-derived extracts or finely ground powders, containing one or more bean-specific lectins, are fed to groups of animals by oral gavage. At various post-inoculation timepoints (e.g, 1, 2, 4, 8, 12, 24 hr), groups of animals are humanely euthanized, intestines removed and sectioned into anatomical regions (e.g, duodendal, ileal, cecal). Gut contents are removed by thorough washing with saline containing protease inhibitors and tissues snap frozen in liquid nitrogen. Lectins are extracted from tissues by standard homogenization methods in extraction buffer containing protease inhibitors and either lectin-specific sugars or 20 mM diaminopropane. Extracted lectins in homogenized supernatants are tested by capture ELISA using lectin-specific antibody pairs and quantitated using a reference curve using commercially purchased purified lectin.

### Example 3

### Compositions for Bean-Specific Lectin In Vitro Activation of Immune Cells

The mitogenic activity of water-soluble bean extracts and finely ground powders from soybean, kidney bean, fava bean and other beans for piscine, avian and various mammalian species (e.g, swine, cattle) has not been reported. Furthermore, bean extracts and finely ground powders contain complex mixtures of native lectins and isoforms that have not be utilized in various combinations to effect the desired biological affect in target species using oral delivery. In contrast, published studies have used highly purified lectins representing one isoform.

In vitro studies using a panel of water-soluble bean-specific extracts or finely ground powders or purified lectins are screened in a checkboard titration studies against a panel of immune cells isolated from avian, piscine and mammalian (e.g, porcine, bovine, canine, feline) species. Results are used to identify lectins and lectin mixtures that are mitogenic for each species.

Lymphocytes are a representative immune system prototypic cell that may be used in in vitro assays for immunological effects of lectins, saponins and other components of natural plant or animal products. Exemplary lymphocytes for representative target species may be obtained and used in the Examples disclosed herein. The effects of various compositions on lymphocyte proliferation is examined using a standard tetrazolium based colorimetric assay (MTT). An Alamar blue-based colorimetric assay gave equivalent results to [³H]-thymidine incorporation assay. The MTT colorimetric assay has also been used in lymphocyte proliferation assays using chicken splenocytes. These and other known assays may be utilized in the methods disclosed herein.

In one non-limiting example, cells from anterior fish kidney are obtained by forcing the aseptically removed tissue through a 100 μm cell strainer into L-15 medium supplemented with 2% fetal bovine serum (FBS), 100 U mL⁻¹ penicillin, 100 μg mL⁻¹ streptomycin, and 10 U mL⁻¹ sodium heparin (Processing medium, PM). The single-cell suspension is removed from the settled tissue fragments and cells are pelleted by centrifugation at 500×g for 10 min at 4° C. Cells are washed by suspension in PM followed by centrifugation as above, suspended in PM and held on ice until use.

Peripheral blood is withdrawn from the caudal vein of anaesthetized fish using a heparinized syringe equipped with a 23 G needle. Blood is immediately diluted in PM and held on ice until use. Leukocyte suspensions (anterior, blood) are layered on 51% Percoll in Hanks Balanced Salt Solution (HBSS) without phenol red, pH 7.2. The cells on Percoll are centrifuged at 500×g for 40 min at 4° C. and the leukocyte fraction is removed from the medium/Percoll interface. Leukocytes are pelleted and washed as described above and then suspended in PM for counting.

The number of viable kidney or blood leukocytes isolated from each fish is determined by trypan blue exclusion (0.1% trypan blue in PM), and the cells are pelleted as described above. Leukocytes are suspended at 2×10⁷ viable cells mL-1 in L-15 supplemented with 5% FBS, 100 U mL⁻¹ penicillin and 100 μg mL⁻¹ streptomycin (Culture medium, CM) and loaded into 96-well tissue-culture plates at 1×10⁶ cells well⁻¹. A volume of 50 μL mL⁻¹ of CM with or without mitogen (control) is added to the wells immediately after cells are plated.

Mitogen concentrations used to stimulate the isolated leukocytes are as follows: 10 μg mL⁻¹ SBA, 10 μg mL⁻¹ PHA-E or PHA-L, 5 μg mL⁻¹ LBL, 20 μg mL⁻¹ LCA or LOA, 10 μg mL⁻¹ ConA, 40 ug mL⁻¹ PSA and 5 μg mL⁻¹ PNA. Mitogen-treated and control wells are replicated in triplicate and plating is performed with the plates on ice. Following plating, leukocytes are incubated in humidified chambers at 10-18° C. (temperature is dependent on fish species, e.g., fresh or coldwater) under atmospheric conditions. The mitogenic response is measured on the fourth day of incubation post-mitogen stimulation using a BrdU-based ELISA at room temperature as described (Gauthier, D. T. 2003). Stimulation index (SI) values are calculated as the replicate mean optical density for a given set of mitogen-treated leukocytes divided by the replicate mean optical density of the associated mitogen free (control) leukocytes. SI values are assigned a relative value according to the following scale: <1 is assigned a value of −− (anti-mitogenic), between 1 and 4.9 is assigned a value of + (weak mitogen), between 5 and 9.9 is assigned a value of ++ (moderate mitogen), and ≧10 is assigned a value of +++ (strong mitogen).

Exemplary results are shown in Tables 3 and 4.

TABLE 3
Immunomodulatory effect of purified lectins on immune cells obtained from fish,
chicken and mammalian species

| Immune | Lectin | | | | | | | | |
|---|---|---|---|---|---|---|---|---|---|
| Cell Source | SBA | PHA-E | PHA-L | LBL | LCA | LOA | Con A | PSA | PNA |
| salmon | +++ | +++ | −−− | +++ | −− | ++ | ++ | −− | +++ |
| trout | + | −− | +++ | +++ | + | −− | +++ | +++ | −− |
| shrimp | −−− | ++ | + | +++ | + | ++ | +++ | +++ | −− |
| avian | +++ | +++ | +++ | −− | + | +++ | ++ | +++ | +++ |
| swine | −− | ++ | −− | ++ | +++ | +++ | + | + | + |
| bovine | + | +++ | +++ | −− | + | ++ | +++ | +++ | + |
| feline | + | ++ | +++ | +++ | −− | −− | + | −− | +++ |
| canine | ++++ | ++ | +++ | +++ | + | −− | + | + | ++ |
| equine | −− | ++ | +++ | −− | +++ | +++ | −− | + | ++ |
| human | ++ | +++ | + | −− | + | +++ | −++ | ++ | + |

−−− anti-mitogenic
+ weak mitogen
++ moderate mitogen
+++ strong mitogen

TABLE 4
Immunomodulatory effect of bean-derived extracts on immune cells
obtained from fish, chicken and mammalian species

| | Bean-derived | | |
|---|---|---|---|
| Immune | extract or finely ground powder | | |
| Cell Source | Soybean | Kidney bean | Lima Bean |
| salmon | +++ | +++ | −− |
| trout | −− | +++ | −− |
| shrimp | ++ | + | + |
| avian | +++ | −−− | + |
| swine | + | −−− | +++ |
| bovine | + | −−− | +++ |
| feline | +++ | +++ | |
| canine | −− | + | +++ |
| equine | + | + | + |
| human | +++ | +++ | + |

−−− anti-mitogenic
+ weak mitogen
++ moderate mitogen
+++ strong mitogen

In another non-limiting example, PBMCs from chicken, rabbit, sheep, horse, calf, swine, dog and cat were prepared. Briefly, blood was aseptically processed within 24 hours by first diluting whole blood with an equal volume of Hank's Balanced Salt Solution (HBSS, Invitrogen) containing 10% fetal bovine serum (FBS, Cambrex). Diluted blood was layered over Histopaque (Sigma) at RT. Gradients were centrifuged for 30 minutes at RT, 600×g. PBMCs were collected from the interface and combined. Collected cells were washed twice with HBSS containing 10% FBS by centrifuging 10 minutes at 250×g. Cell pellets were combined. For all species except chicken, a red blood cell (RBC) lysis step was done to lyse residual RBC. For chicken, residual RBC were left in the preps, as they are nucleated and cannot be lysed easily. After a final wash in HBSS containing 10% FBS, cells were counted and resuspended in RPMI (Gibco) containing 10% FBS, pen/strep (Gibco), HEPES (Gibco) and Glutamax (Gibco). Cells were plated in triplicate (5×10⁵ cells/well) in 96 well U-bottom plates with purified lectins ConA, PHA-E+L, PHA-E, PHA-L, SBA (Vector Labs), LBL (EY Labs) at the indicated concentrations, Oralject extracts (Formula 1 (Avaire), Formula 2 (A-03), and Formula 7 (A-16) or bean extracts (ground soybeans, ground lima beans, ground white kidney bean, ground dark red kidney beans) at various dilutions. Plates were incubated at 37±2° C. (rabbit, horse, calf, swine, sheep, dog, cat) or 41±2° C. (chicken) for 1-4 days.

For mouse splenocyte preparation, spleens from BALB/c mice were aseptically harvested. Single cell suspensions were generated by pushing the spleen tissue through 70 μm cell strainers into HBSS containing 10% FBS. Cells in suspension were collected in the HBSS, combined and centrifuged at 250×g to pellet cells. The cell pellet was resuspended in RBC lysis solution and centrifuged 10 minutes at 250×g to remove RBC. After 2 additional washes in HBSS containing 10% FBS, cells were counted and resuspended in RPMI containing 10% FBS, pen/strep, HEPES and Glutamax. Cells were plated in triplicate (5×10⁵ cells/well) in 96 well U-bottom plates with purified lectins, Oralject extracts or bean extracts and incubated at 37±2° C. for 1-4 days.

Trout head kidney cells were prepared by pushing the tissue through 70 μm cell strainers into HBSS containing 10% FBS. After further dilution of the suspension, several 1 ml aliquots were each treated with 1 ml RO/DI water to lyse RBC. Immediately after the addition of the water, 48 ml of HBSS containing 10% FBS was added to dilute out the released nuclear material. All samples were centrifuged 10 minutes at 250×g to pellet the remaining WBC. WBC pellets were combined and washed with HBSS containing 10% FBS. Cells were counted and and resuspended in MEM containing 10% FBS, and pen/strep. Cells were plated in triplicate (5×10⁵ cells/well) in 96 well U-bottom plates with purified lectins, Oralject extracts or bean extracts and incubated at 15±2° C. for 1-4 days.

Proliferation in response to the lectins or extracts was read by visual microscopic observation, and by colorimetric observation by the addition of standard tetrazolium proliferation reagents (CellTiter 96 AQᵤₑₒᵤₛ Cell Proliferation Reagent, Promega or WST-1, Roche) directly to the cultured cells in plates. After the addition of the reagent, plates were incubated for an additional 4 hours at the indicated temperatures (8 hours for trout HK cells). Color development was read on a standard plate reader. Stimulation Index (reading from lectin treated cells divided by reading from media control cells) was plotted against lectin concentration. Representative data are shown in FIG. 7 and in Tables 5-7.

The results (FIG. 7) showed a dose-dependent and cell species-dependent response to PHA-E+L stimulation, with maximum proliferation of chicken PBMC, calf PBMC and trout head kidney immune cells at approximately 100, 1, and 10 μg/ml PHA-E+L, respectively. Results further demonstrated that the composite lectin stimulatory profile was species dependent (Table 5).

TABLE 5
Stimulation of Immune Cells from Various Species by Purified Lectins

| Cell Type | ConA | PHA-E + L | LBL | SBA |
|---|---|---|---|---|
| Sheep PBMC | 5 ug/ml | 5 ug/ml | 100 ug/ml | 50 ug/ml |
| Chicken PBMC | 1 ug/ml | 1 ug/ml | 100 ug/ml | Negative |
| Trout HK Cells | 0.1 ug/ml | 0.1 ug/ml | Negative | Negative |
| Mouse | 5 ug/ml | 5 ug/ml | Negative | Negative |
| Splenocytes | | | | |
| Calf PBMC | 0.5 ug/ml | 0.1 ug/ml | Negative | 50 ug/ml |
| Swine PBMC | 5 ug/ml | 0.1 ug/ml | Negative | 50 ug/ml |
| Feline PBMC | 5 ug/ml | 5 ug/ml | 5 μg/ml | 1 μg/ml |

Values indicate lowest concentration of lectins required for stimulation response above background

For example, trout HK cells were stimulated by Con A and PHA E+L at 0.1 μg/ml. but were refractory to LBL and SBA stimulation. In contrast, chicken and sheep PMBC required a 10- or 50-fold higher amount, respectively, of Con A or PHA-E+L compared to trout HK cells in order to become stimulated. In addition, results show that some lectins (e.g., Con A, PHA-E+L) stimulated all cell species tested, whereas other lectins (e.g., LBL, SBA) only stimulated a subset of cell species.

Thus, the data show that immunoactive compositions for improved oral delivery of vaccines for aquatic species should preferably be comprised of whole bean, pea, nut or other plant extracts, homogenates or ground powders that contain relatively low levels (e.g., 0.1-1.0 μg/ml) of the specific lectins ConA or PHA Furthermore, immunoactive compositions for improved oral delivery of vaccines for avian species species should preferably be comprised of whole bean, pea, nut or other plant extracts, homogenates or ground powders that contain relatively moderate amounts (e.g., 1.01-5.0 μg/ml) of the lectins ConA or PHA. Furthermore, immunoactive compositions for improved oral delivery of vaccines for terrestrial mammalian species (e.g, livestock, companion animals, humans) should preferably be comprised of whole bean, pea, nut or other plant extracts, homogenates or ground powders that contain relatively high amounts of the lectins ConA or PHA The data also demonstrate that immunoactive compositions for oral delivery of vaccines to terrestrial species can be enhanced or improved through the addition of other whole bean, pea, nut or other plant extracts, homogenates or ground powders that contain relatively higher amounts of the lectins LBL and/or SBA. Finally, the data also demonstrate that compositions for oral delivery of biotherapeutics to aquatic and terrestrial species can be enhanced or improved through the elimination of other whole bean, pea, nut or other plant extracts, homogenates or ground powders that contain mitogenic lectins.

Various Oralject extracts were also demonstrated to contain immune cell stimulatory activity (Table 6).

TABLE 6
Stimulation of Immune Cells from Various Species
by Oralject Extracts (Colorimetric Data)

| Cell Type | Formula 1 | Formula 2 | Formula 7 |
|---|---|---|---|
| Sheep PBMC | 640 | 1,280 | 1,280 |
| Chicken PBMC | 1,024 | 4,096 | 1,024 |
| Trout HK Cells | Negative | 6,400 | 800 |
| Mouse Splenocytes | Negative | 100 | 100 |
| Calf PBMC | 40 | 5120 | 5120 |
| Swine PBMC | Negative | 5120 | 1280 |

Values indicate the reciprocal of the highest dilution at which stimulation occurred (response above background)

These results clearly demonstrate that the lectin containing components in Formulas 1, 2 and 7 are bioactive and bioavailable in their respective formulations based on their ability to stimulate immune cells from numerous aquatic and terrestrial species. Formula 2 and 7 were mitogenic against all cell species tested, whereas formula 1 only stimulated a subset of cell species (e.g., sheep, chicken, and calf). The highest titratable mitogenic activity of formula 2 was observed against trout HK cells, whereas formula 7 had the highest tritratable mitogenic activity against calf PBMC and formula 1 had the highest titratable stimulatory activity against chicken PBMC. The results further show that the immune cell compartment source also influenced formula mitogenic activity. For example, formula 1 was mitogenic for immune cells of blood origin (PBMC) but was not mitogenic for immune cells derived from head kidney or splenocytes.

Thus, the data demonstrate that immunoactive compositions for improved oral delivery of vaccines to the intestinal immune tissue compartments for aquatic species should preferably be comprised of whole bean, pea, nut or other plant extracts, homogenates or ground powders and other ingredients contained in Formula 2. Furthermore, the data demonstrate that immunoactive compositions for improved oral delivery of vaccines to the systemic immune compartment for avian species should preferably be comprised of whole bean, pea, nut or other plant extracts, homogenates or ground powders and other ingredients contained in Formula 2. Furthermore, the data demonstrate that immunoactive compositions for improved oral delivery of vaccines to the systemic immune compartment for livestock species should preferably be comprised of whole bean, pea, nut or other plant extracts, homogenates or ground powders and other ingredients contained in Formula 2 or Formula 7. Furthermore, the data demonstrate that immunoactive compositions for improved oral delivery of vaccines to the immune tissue compartment for terrestrial species should preferably be comprised of whole bean, pea, nut or other plant extracts, homogenates or ground powders and other ingredients contained in Formula 2 or Formula 7. Furthermore, the data demonstrate that compositions for improved oral delivery of biotherapeutics to aquatic and terrestrial species should preferably be comprised of whole bean, pea, nut or other plant extracts, homogenates or ground powders and other ingredients contained in Formula 1.

The results further demonstrate that the bean extracts differ in their ability to stimulate immune cells from different species (Table 7).

TABLE 7
Stimulation of Immune Cells from Various Species by Bean Extracts

| | | | White | Dark Red |
|---|---|---|---|---|
| | | | Kidney | Kidney |
| | | | Bean | Bean |
| Cell Type | Soybean | Lima Bean | (WKB) | (DRKB) |
| Sheep PBMC | 640 | 640 | 1,280 | 1,280 |
| Chicken PBMC | Negative | Negative | 40,960 | 40,960 |
| Trout HK Cells | Negative | Negative | 8,000 | 8,000 |
| Mouse Splenocytes | Negative | Negative | Negative | Negative |
| Calf PBMC | Negative | Negative | >81,920 | >81,920 |
| Swine PBMC | Negative | Negative | >81,920 | >81,920 |

Values indicate the reciprocal of the highest dilution at which stimulation occurred (response above background)

For example, soybean and lima bean extracts have relatively low and species-restricted mitogenic activity compared to white kidney bean and dark red kidney beans. White and dark red bean extracts have potent mitogenic activity against 83% (⅚) of the cell species tested compared to only 17% (⅙) of the cell species tested for soybean and lima bean extracts. As observed with the Oralject formulas (F1, F2, F7) (Table 6), the immune cell compartment source also influenced bean extract mitogenic activity. For examples, splenocytes were refractory to stimulation with all four bean extracts, whereas sheep PBMC were stimulated by all four bean extracts. Lastly, as observed with the Oralject formulas (F1, F2, F7) (Table 6), the results show that the composite bean extract stimulatory profile is species dependent.

Thus, the data demonstrate that immunoactive compositions for improved oral delivery of vaccines to the intestinal immune compartment for aquatic species should preferably be comprised of extracts, homogenates or ground powders from white or dark red kidney bean. Furthermore, the data demonstrate that immunoactive compositions for improved oral delivery of vaccines to the systemic immune compartment for terrestrial species should preferably be comprised of extracts, homogenates or ground powders from white or dark red kidney bean. Furthermore, the data demonstrate that compositions for improved oral delivery of biotherapeutics to aquatic or terrestrial species should not contain highly stimulatory lectins from white or dark red kidney bean.

Collectively, these results show that relative proportions of plant or non-plant extracts, homogenates, finely ground powders or derivatives may be selected to optimize the content of one or more mitogenic lectins of use in the composition for oral delivery of bioactive agents to aquatic and terrestrial species.

### Example 4

### Compositions for Bean-Specific Lectin In Vitro Stimulation of Enterocyte CCK Release.

The intestinal endocrinal cell line STC-1 (American Type Culture Collection, Manassas, Va.), is grown in RPMI-1640 medium supplemented with 5% (v/v) fetal calf serum, 2 mM glutamine and antibiotics (100 units/ml penicillin and 50 uM streptomycin) in a humidified C02 incubator at 37° C. Cells are plated in microtiter wells at 1×10⁵ cells/well in triplicate and incubated overnight at 37° C., 5% CO2. Culture medium is removed and plates incubated at 37° C. with Kreb's Ringer bicarbonate buffer and serial dilutions of bean-derived extracts containing different amounts and types of lectins. Following incubation for 1 hr, plates are placed on ice and medium collected and centrifuged at 4° C. for 5 min at 100 g to remove any residual cells. The medium is frozen at −25° C. until the CCK assay is performed using assays previously known in the art.

### Example 5

### Compositions for Fatty Acid In Vitro Modulation of Immune Cells

Bean-derived fatty acid activation of immune cells from piscine, avian and various mammalian species (e.g, swine, cattle) has not previously been reported. In vitro studies using a panel of bean-derived, n-3 and n-6 polyunsaturated fatty acids are screened in checkboard titration studies against a panel of immune cells isolated from avian, piscine and mammalian (e.g, porcine, bovine, canine, feline) species. Results are used to identify compositions comprising n-3 and n-6 fatty acids that induce immune cell proliferation or cytokine expression for in target species.

Lymphocyte proliferation assays may be performed as described in Example 3. Cytokine expression may be determined by a variety of standard techniques known in the art, such as ELISA (Assay Designs, Ann Arbor, Mich.), PCR assay (SuperArray Bioscience Corp., Frederick, Md.), Meso Scale Discovery analysis (Gaithersburg, Md.). Alternatively, cytokine activity may be assayed by cellular proliferation of primary cell cultures or adapted cell lines (e.g., eBioscience, San Diego, Calif.), Standard protocols are available for assaying cytokine-induced proliferation, cytokine-induced killing, protection against viral effects or cytokine-induced cytokine production using in vitro cell assays.

In one exemplary embodiment, cells from anterior fish kidney are obtained by forcing the aseptically removed tissue through a 100 μm cell strainer into L-15 medium supplement with 2% fetal bovine serum (FBS), 100 U mL⁻¹ penicillin, 100 μg mL⁻¹ streptomycin, and 10 U mL⁻¹ sodium heparin (Processing medium, PM). The single-cell suspension is removed from the settled tissue fragments and cells are pelleted by centrifugation at 500×g for 10 min at 4° C. Cells are washed by suspension in PM followed by centrifugation as above, suspended in PM and held on ice until use.

Peripheral blood is withdrawn from the caudal vein of anaesthetized fish using a heparinized syring equipped with a 23 G needle. Blood is immediately dilution in PM and held on ice until use. Leukocyte suspensions (anterior, blood) are layered on 51% Percoll in Hanks Balanced Salt Solution (HBSS) without phenol red, pH 7.2. The cells on Percoll are centrifuged at 500×g for 40 min at 4° C. and the leukocyte fraction is removed from the medium/Percoll interface. Leukocytes are pelleted and washed as described above and then suspended in PM for counting.

The number of viable kidney or blood leukocytes isolated from each fish is determined by trypan blue exclusion (0.1% trypan blue in PM), and the cells are pelleted as described above. Leukocytes are suspended at 2×10⁷ viable cells mL⁻¹ in L-15 supplemented with 5% FBS, 100 U mL⁻¹ penicillin and 100 μg mL⁻¹ streptomycin (Culture medium, CM) and loaded into 96-well tissue-culture plates at 1×10⁶ cells well⁻¹.

A volume of 50 μL mL⁻¹ of CM with or without purified n-3 and n-6 polyunsaturated fatty acids (control) is added to the wells immediately after cells are plated. Purified n-3 and n-6 polyunsaturated fatty acids concentrations used to stimulate the isolated leukocytes range from 1 nM to 100 μM. N-3 and n-6 polyunsaturated fatty acid-treated and control wells are replicated in triplicate and plating is performed with the plates on ice. Following plating, leukocytes are incubated in humidified chambers at 10° C.-18° C. (temperature is dependent on fish species, e.g., fresh or coldwater) under atmospheric conditions. The mitogenic response is measured on the fourth day of incubation post-mitogen stimulation using a BrdU-based ELISA at room temperature as described (Gauthier, D. T. 2003). Stimulation index (SI) values are calculated as the replicate mean optical density for a given set of n-3 and n-6 polyunsaturated fatty acid-treated leukocytes divided by the replicate mean optical density of the associated n-3 and n-6 polyunsaturated fatty acid-free (control) leukocytes.

An exemplary result is shown in Table 8.

TABLE 8
Proliferation of fish lymphocytes using bean-derived, purified n-3 and n-6
polyunsaturated fatty acids.

| | Bean-derived source and fatty acid type* | | | | |
|---|---|---|---|---|---|
| Immune | | Soybean/ | Kidney | Kidney | Lima |
| Cell Source | Soybean/n-3 | n-6 | bean/n-3 | bean/n-6 | bea/n-6 |
| salmon | 4 | 16 | 25 | 2 | 8 |
| trout | 16 | 14 | 50 | 4 | 8 |
| shrimp | 2 | 7 | 100 | 7 | 8 |
| avian | 1 | 4 | 75 | 9 | 8 |
| swine | 1 | 80 | 24 | 12 | 8 |
| bovine | 1 | 34 | 24 | 45 | 8 |
| feline | 1 | 26 | 56 | 25 | 8 |
| canine | 35 | 68 | 56 | 20 | 8 |
| equine | 23 | 23 | 80 | 15 | 80 |
| human | 8 | 5 | 200 | 12 | 25 |

*stimulation index (S.I.) = proliferation + fatty acid/proliferation + media

In another exemplary embodiment for PUFA, a murine macrophage cell line, designated RAW264.7 (ATCC), was plated in 96 well flat bottom plates at 10⁵ cells/well. Cells were incubated overnight at 37±2° C. After overnight incubation, media was removed from wells, and five different PUFAs (linoleic acid [LA], arachidonic acid [AA], eicosapentaenoic acid [EPA], docosapentaenoic acid [DPA], docoahexaenoic acid [DHA], all from Nu-Chek Prep) diluted in ethanol were added at the indicated concentrations in duplicate. Cells were incubated with PUFA 24 hours at 37±2° C. After 24 hour incubation, LPS (Sigma) was added to cells at 1 μg/ml, and plates were incubated 8 hours at 37±2° C. Following incubation with LPS, supernatants were sampled and tested for nitric oxide. Ethanol carrier control was tested at the same ethanol dilution as used in the PUFA treatments.

Supernatants were tested for NO using a commercial kit (R&D Systems). This kit uses a nitrate reductase step to reduce all nitrate to nitrite. Briefly, supernatants and nitrate standards were added in duplicate to a 96 well plate. Nitrate reductase and NADH were added, and the plate was incubated 30 minutes at 37±2° C. After incubation, Griess reagents I and II were added sequentially, and plate was incubated 10 minutes at RT. The plate was read for color development at 540 nm (690 nm reference wavelength). Data is shown as percent inhibition of NO production vs. PUFA concentration (FIG. 8).

Representative data are shown in FIG. 8. These results clearly demonstrate that PUFA composition influences the degree of immunomodulatory activity. For examples, the N-6 PUFAs tested (LA, AA) have marginal to no immunomodulatory activity, whereas the N-3 PUFAs tested (EPA, DPA) have high immunomodulatory activity. The N-3 PUFA, DPA, is 10-100 fold more effective at inhibiting PHA-induced proliferation that the N-6 PUFA, AA. DPA inhibited proliferation by 40% at 1 μM, whereas 100 μM AA is required to inhibit proliferation to a similar level.

Thus, the data demonstrate that immunostimulatory compositions for improved oral delivery of vaccines to aquatic and terrestrial species should preferably be comprised of N-6 PUFAs. In contrast, the data demonstrate that immunosuppressive or immunotolerant compositions for improved oral delivery of therapeutics (e.g, antibiotics, drugs) to aquatic and terrestrial species should preferably be comprised of N-3 PUFAs.

Collectively, these results show that relative proportions of plant or non-plant extracts, homogenates, finely ground powders or derivatives may be selected to optimize the content of one or more fatty acids in the composition for either immunostimulatory activity or immunotolerant or immunosuppressive activity.

### Example 6

### Compositions for Isoflavone In Vitro Modulation of Immune Cells

Bean-derived isoflavone acid activation of immune cells from piscine, avian and various mammalian species (e.g, swine, cattle) has not previously been reported. In vitro studies using a panel of isoflavones derived from plant sources are screened in checkboard titration studies against a panel of immune cells isolated from avian, piscine and mammalian (e.g, porcine, bovine, canine, feline) species. Assays are performed as described in Examples 1-5 above. Results are used to identify compositions comprising specific isoflavones that induce immune cell proliferation or cytokine expression for each species.

In one exemplary embodiment, chicken intestinal epithelial lymphocytes (IELs) are isolated similar to published methods. Birds are euthanized and the intestinal tract is removed longitudinally from duodenal loop to iliocecal junction. The fat and blood vessels on the serosal surface are removed, the intestines are cut into small pieces and washed several times with phosphate buffered saline (PBS) to remove detritus. The intestinal pieces are transferred to a beaker containing pre-warmed 2 mM DTT and incubated in a water bath at 39° C. for 15 min with occasional shaking to remove the intestinal mucus. The cloudy suspension is discarded and the DTT treatment is repeated with fresh solution. The tissue segments are washed with PBS and transferred to a fresh beaker containing 1 mM EDTA, stirred gently for 30 min on a magnetic stirrer at room temperature. The supernatant is allowed to settle for 15 min to remove clumps of epithelial cells. The supernatant containing cells is filtered two times through a pre-soaked nylon wool column. The filtrate is centrifuged at 1000×g for 10 min. and the pellet is suspended in RPMI-1640 medium.

The cell suspension is further purified by density gradient centrifugation on histopaque. The cellular band at the interface between medium and histopaque is collected and washed with PBS at 1000×g for 10 min. The pellet is suspended in RPMI medium. The cell count and viability is determined by trypan blue dye exclusion method.

The final IELs concentration is adjusted to 2×10⁷ viable cells/ml and cells are loaded into 96-well tissue-culture plates at 1×10⁶ cells per well. A volume of 50 μL mL⁻¹ of RPMI with or without purified isoflavones is added to the wells immediately after cells are plated. Purified isoflavone concentrations used to stimulate the isolated leukocytes range from 1 nM to 10 0 μM. Purified isoflavone-treated and control wells are replicated in triplicate and plating is performed with the plates on ice. Following plating, leukocytes are incubated in humidified chambers at 39° C., 5% C02. The production of cytokines IL-4, IL-10 and IFN-γ is measured by conventional RT-PCR using published methods known to those in the art.

An exemplary result is shown in Table 9.

TABLE 9
Immunomodulatory effect of purified bean-derived, isoflavones
on cytokine expression in chicken lymphocytes.

| | Purified Isoflavone - Cytokine Secretion Profile | | |
|---|---|---|---|
| Immune cell source | Gentisein | Daidzein | Biochanin A |
| salmon | IL-10 | IFN-gamma | IL-4 |
| trout | IL-4, IL-10 | IFN-gamma | None |
| shrimp | None | IFN-gamma | None |
| avian | IFN-gamma | None | IL-4 |
| swine | IL-4 | None | None |
| bovine | IL-10 | IL-10 | None |
| feline | None | IL-4 | IL-4, IL-10 |
| canine | None | None | IL-4, IL-10 |
| equine | None | None | IL-10 |
| human | IL-10 | IFN-gamma | none |

determined by RT-PCR

In another exemplary embodiment, the ability of isoflavones to modulate the ability of calf cells to generate IFN-gamma in response to PHA-E+L stimulation was tested. Calf PBMC were isolated as described in Example 3. Cells were plated in triplicate (5×10⁵ cells/well) in 96 well U-bottom plates. Isoflavones (genistein, daidzein, biochanin A) (Sigma) at the indicated concentrations and PHA-E+L (1 ug/ml) were added to the cells and incubated at 37±2° C. for 3 days. After incubation, supernatants were sampled. Supernatants from identical treatment wells were combined and stored frozen at −18±5° C. until tested for IFN-gamma.

Supernatant samples were tested for bovine IFN-gamma using a commercial kit (Thermo Scientific). Briefly, plates were coated overnight with capture antibody in carbonate coating buffer. Plates were washed and blocked for 1 hour at RT. Standards were diluted and samples and standards were added in duplicate to coated, blocked plates and incubated 1 hour at RT. Plates were washed and treated with biotinylated detection antibody for 1 hour at RT. Plates were washed and treated with streptavidin-HRP for 30 minutes at RT. Plates were washed and substrate was added for 20 minutes at RT in the dark. Following incubation, stop solution was added to each well, and plates were read at 450 nm (550 nm reference wavelength). Data was plotted as Stimulation Index (IFN-gamma concentration of sample treated with isoflavone divided by IFN-gamma concentration of sample treated with PHA only) vs. isoflavone concentration. Since isoflavones dilutions were made in DMSO, the DMSO carrier was tested at the corresponding concentrations to determine the carrier effect.

The results (FIG. 9) clearly demonstrate that the isoflavone effect on IFN-gamma production was both isoflavone type and concentration dependent. For example, 10 nM or 100 nM genistein and daidzein resulted in a pronounced decrease in stimulation indices compared to DMSO control, whereas 1 μM of genistein, daidzein or biochanin A resulted in a pronounced increase in stimulation indices compared to DMSO control. At the highest isoflavone concentration tested (100 μM), both daidzein and biochanin A also had an inhibitory effect, whereas no effect was seen at this same concentration with genistein.

Thus, the data demonstrate that immunactive compositions for oral delivery of vaccines to aquatic and terrestrial species can be generated that preferentially induce either a T-helper 1 or T-helper 2 immune response. For example, to generate a T-helper 2 response (low IFN-gamma production) relatively low amounts (e.g. 10 nM or 100 nM) of specific isoflavones (genistein, daidzein) are preferably used, whereas to preferentially generate a T-helper 1 response (high IFN-gamma production) relatively moderate amounts (e.g, 1-5 μM) of specific isoflavones (genistein, daidzein or biochanin) are used.

In yet another exemplary embodiment, the ability of isoflavones to modulate the production of LPS-induced NO generation in RAW264.7 cells was investigated. The cells were plated in 96 well flat bottom plates at 10⁵ cells/well. Cells were incubated overnight at 37±2° C. After overnight incubation, media was removed from wells, and isoflavones diluted in DMSO carrier were added at the indicated concentrations in duplicate. DMSO carrier control was tested at the same ethanol dilution as used in the isoflavone treatments. Cells were incubated with isoflavones for 1 hour at 37±2° C. After 1 hour incubation, LPS (Sigma) was added to cells at 1 μg/ml, and plates were incubated overnight at 37±2° C. After overnight incubation, supernatants were sampled and tested for nitric oxide concentration as described in Example 5.

The results (FIG. 10) clearly show that all three isoflavones inhibited LPS-induced NO generation by a maximum of 30-40%. Maximum inhibition for all three compounds was observed at the highest isoflavone concentration tested (100 μM). However, the inhibitory effect was both dose- and isoflavone-dependent. For example, no inhibition was observed at the lowest concentrations tested. Biochanin A had an approximately 10-fold higher inhibitory activity at 25 μM compared to daidzein or genistein.

Thus, the data demonstrate that immunactive compositions for oral delivery of vaccines to aquatic and terrestrial species can be generated that preferentially induce a pro-inflammatory, T-helper 2 response (NO production). For example, to generate a proinflammatory, T-helper 2 response, relatively high (100 μM) amounts of isoflavones are used in order to induce NO production, which is proinflammatory and also possesses a T-helper 2 immunosuppresive effect. This effect can be be further enhanced through compositions which contain relatively higher amounts of the isoflavone Biochanin A which has higher activity compared to other isoflavones such as daidzein or genistein.

### Example 7

### Compositions for Saponins In Vitro Modulation of Immune Cells

Bean derived saponin modulation of immune cells has not been previously disclosed. In this example, the effects of soyasaponins on LPS-induced NO generation were demonstrated. RAW264.7 cells were plated in 96 well flat bottom plates at 10⁵ cells/well. Cells were incubated overnight at 37±2° C. After overnight incubation, media was removed from wells, and classes of saponin compounds (Group A saponins, Group B saponins and Sapogenol B, provided by Dr. Mark Berhow, USDA-ARS; these compounds can also be purchased commercially from Organic Technologies) diluted in DMSO carrier were added at the indicated concentrations in duplicate. Cells were incubated with saponins 1 hour at 37±2° C. After 1 hour incubation, LPS (Sigma) was added to cells at 1 μg/ml, and plates were incubated overnight at 37±2° C. After overnight incubation, supernatants were sampled and tested for nitric oxide as described in Example 5.

Results (FIG. 11) clearly demonstrated that the inhibitory effect on LPS-induced NO production was both saponin type- and dose-dependent. No inhibition was detected by the Group A saponin, whereas the Group B saponins and sapogenol B inhibited LPS-induced NO production. Zero to minimal inhibition was detected by Group B saponins and sapogenol B at the lowest concentrations, whereas at the highest concentration tested (100 μg/ml) both compounds showed approximately 15-25% inhibition. Sapogenol B was the most potent inhibitor.

Thus, the data demonstrate that immunactive compositions for oral delivery of vaccines to aquatic and terrestrial species can be generated that preferentially suppress a pro-inflammatory, T-helper 2 response (NO production) and preferentially induce a T-helper 1 response. For example, to suppress a proinflammatory T-helper 2 response and induce a T-helper 1 response, relatively high concentrations (100 μg/ml) of Group B saponins and sapogenol B can be preferentially added to the compositions. This effect can be be further enhanced through compositions which contain relatively higher amounts of sapogenol B which has higher potency compared to other Group B saponins.

### Example 8

### Effect of Different Formulations on In Vitro Hemagglutination Activity

Compositions for bean-specific agglutination of red blood cells. As previously discussed, the multimeric structure of lectins contained in bean extracts can confer cell agglutination. Although many bean extracts test positive for cell agglutination, some may bind cells and not cause agglutination. Studies were conducted to assess the hemagglutination activity of two different formulas. Formula 1 (ingredients described in Example 1) contained soybeans and lima beans and Formula 2 (ingredients described in Example 1) contained soybeans, lima beans, white beans, and red kidney beans. These formulas were used for hemagglutination studies using chicken and rabbit red blood cells.

An exemplary result is shown in Table 10.

TABLE 10
Hemagglutination activity of formulas containing
different compositions of bean extracts.

Titer of Hemagglutination Activity*

| Oralject | Chicken red blood | |
|---|---|---|
| Formulation | cells | Rabbit red blood cells |
| Formula 1 | <12 | 3072 |
| Formula 2 | 768 | 6144 |

The results (Table 10) showed that the formula bean composition influences both the specificity and the strength of hemagglutination activity. Formula 1 failed to agglutinate chicken red blood cells whereas Formula 2 showed positive hemagglutination to chicken RBCs. This result indicates that the specificity of chicken RBC binding is due to the lectin PHA-E that is present in both white and red kidney beans found in the Formula 2 composition but absent from the Formula 1 composition. Therefore, the sugar residues present on the surface of chicken RBC bind to PHA-E lectin, whereas SBA and LBA lectins failed to recognize chicken RBC sugar residues. In contrast, both Formula 1 and 2 compositions showed positive hemagglutination to rabbit RBCs. Thus, the sugar residues present on the surface of rabbit RBC are recognized by SBA and/or LBA lectins found in soybean and lima bean extracts, respectively. Finally, the hemagglutinatiuon activity observed with rabbit RBCs was increased through the addition of PHA-E lectin found in white and red kidney bean extracts, since the HA titers were two-fold higher using Formula 2 versus Formula 1. These data further support the concept that the activities and/or properties of oral delivery compositions containing bean extracts or finely ground powders can be specifically modulated through the addition or omission of specific lectin containing bean extracts or finely ground powders to affect binding specificity and binding strength to bioagents used for oral delivery,

### Example 9

### Effect of Different Formulations on In Vivo Immune Response to Vaccine Antigen

Compositions for bean-specific activation of antigen-specific antibody reponses in chickens. Two Oralject™ formulations were evaluated in this study. The purpose of the study was to determine if a mucosal response to Newcastle Disease Virus (NDV) can be detected in birds when vaccinated with a complex of inactivated NDV non-purified antigens formulated with Oralject™ in the absence or presence of adjuvant (acrylic polymer) and immunostimulatants (Quil A or LT) via oral gavage. One base formulation was designated F1 and the other formulation was designated F2. Both Oralject™ formulations were individually weighed and dispensed into storage containers the day prior to each vaccination and stored at room temperature overnight. Oralject™ amounts used for each vaccination are shown in Table 11. The increased amount of F1 and F2 used on Day 14 and Day 28 was based on the higher mean body weight of birds at these timepoints.

TABLE 11
ORALJECT ™ AMOUNTS PER GAVAGE VACCINATION

Oralject ™ Formulation
Amount (grams)^{a}

| | Formula 1 | Formula 2 |
|---|---|---|
| Day of Study | (Per Bird) | (Per Bird) |
| Day 0 | 2.4 | 2.4 |
| Day 14 | 4.2 | 4.2 |
| Day 28 | 7.1 | 7.1 |

^{a}Amount of Oralject ™ oral gavaged at each AM and PM vaccination

The liquid component of each vaccine, containing inactivated NDV antigens, adjuvant and immunostimulant, was prepared and dispensed into aliquots the day prior to each vaccination and held at 2-7° C. until vaccination or prepared the same day prior to each vaccination. The Oralject™ formulations were blended by vortexing with the liquid preparation (containing NDV antigens, adjuvant and immunostimulant) for each treatment prior to each gavage vaccination. The liquid, oil and Oralject™ components were aliquoted for each treatment group so there was one set of each component available for the morning (am) gavage and another set available for the afternoon (pm) gavage. The target antigen concentrations for the study are described in Table 12 below. Calculation of the HN target dose was based on an HN specific ELISA quantitation of the Newcastle Disease Virus (NDV) stock material derived from allantoic fluid.

TABLE 12
Target HN Concentration Per Gavage Vaccination

| | HN |
|---|---|
| | (μg/dose) |
| Day of Study | Oral Gavage Groups^{b} |
| Day 0 | 10.6 |
| Day 14 | 17.5 |
| Day 28 | 29.3 |

^{a}Antigen concentration at each AM and PM vaccination
^{b}T3-T8 only

Leghorn specific-pathogen-free chicks (approximately 25-30 days of age) were fasted for 12-16 hours (overnight) prior to vaccine administration. On day 0, birds (n=4/group) were vaccinated according to the treatments described in Table 13 by oral gavage (OG) with half the dose.

TABLE 13
Experimental Design

| Trt. | Treatment | # | | Day of | IgG |
|---|---|---|---|---|---|
| No. | Group Description | birds | Route | vaccination¹ | sample |
| T1 | Formula 1 (control) | 4 | OG | 0, 14 and 28 | Day 42 |
| T2 | Formula 2 (control) | 4 | OG | 0, 14 and 28 | Day 42 |
| T3 | Formula 1 + NDV | 4 | OG | 0, 14 and 28 | Day 42 |
| T4 | Formula 2 + NDV | 4 | OG | 0, 14 and 28 | Day 42 |
| T5 | Formula 1 + NDV + acrylic | 4 | OG | 0, 14 and 28 | Day 42 |
| | polymer/Quil A | | | | |
| T6 | Formula 2 + NDV + acrylic | 4 | OG | 0, 14 and 28 | Day 42 |
| | polymer/Quil A | | | | |
| T7 | Formula 1 + NDV + acrylic | 4 | OG | 0, 14 and 28 | Day 42 |
| | polymer/LT | | | | |
| T8 | Formula 2 + NDV + acrylic | 4 | OG | 0, 14 and 28 | Day 42 |
| | polymer/LT | | | | |

Birds were orally administered vaccines twice on each day, with vaccinations occurring approximately 4 hours apart.

Birds in groups T1-T8 were administered the second half of the vaccine by OG approximately 4 hours post vaccination. Normal feeding of all birds was resumed approximately 2 hours post second inoculation. On Day 13, birds were fasted for 12-16 hours (overnight). On Day 14, birds were vaccinated using the same methods and treatments as on Day 0. On day 27, birds were fasted for 12-16 hours (overnight). On Day 28, birds were vaccinated using the same methods and treatments as on Days 0 and 14. On day 42, birds were euthanized and a section of the intestine on each bird (n=4/group) was excised to encompass the distal ilieum, cecal tonsils, and colorectrum. Samples were placed in a tube containing buffer (DPBS+0.1% Tween 20+0.1 mg/ml soybean trypsin inhibitor), vortexed and placed on ice until further testing. Samples were cut and minced into small pieces and placed in 24-well tissue culture plates (2.0 ml/well) containing cell culture medium and antibiotics. After 48 hrs. approximately 0.5 ml of supernatant was removed and microfuged at high speed for 2 minutes to pellet any tissue debris. Cell-free supernatant was collected and stored at −80° C. until testing by IgG-specific HN ELISA as described above.

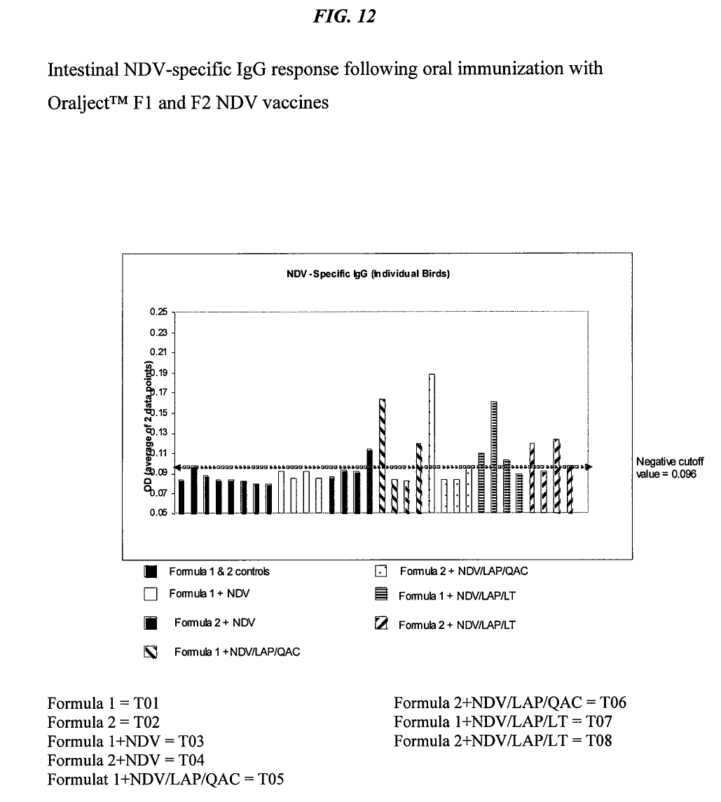

Results (FIG. 12) show that control birds orally immunized with Formula 1 (T1) and Formula 2 (T2) alone were used to establish the negative cutoff level (set at 0.096 OD). The seroconversion percentage (defined herein as # of birds with an average OD value higher than the negative cutoff level divided by the total # of birds in treatment group) was 0% in T3 and 25% in T4. Addition of acrylic polymer adjuvant and Quil A immunostimulant to Formula 1 resulted in an increase to 50% seroconversion (T5) compared to Formula 1+NDV alone (T3), whereas addition of acrylic polymer adjuvant and LT immunostimulant to Formula 1 resulted in a further increase to 75% seroconversion (T7) compared to Formula 1+NDV alone (T3). Addition of acrylic polymer adjuvant and Quil A immunostimulant to Formula 2 failed to result in an further increase in seroconversion rate (T6) compared to Formula 2+NDV alone (T4), whereas addition of acrylic polymer adjuvant and LT immunostimulant to Formula 2 did provide a modest increase in seroconversion rate to 50% (T8) compared to Formula 2+NDV alone. These results support the concept that Oralject™ formulations carrying complex antigen mixtures can induce antigen-specific IgG mucosal responses following oral delivery to birds. In this particular study, the number of birds with detectable NDV-specific intestinal IgG titers (e.g., seroresponders) was highest (75%) in T7. This group received NDV vaccine in Oralject™ Formula 1 containing acrylic polymer adjuvant and LT immunostimulatant. In this particular study, the highest NDV-specific intestinal IgG individual titer was detected in a T6 bird that received NDV vaccine in Formula 2 containing acrylic polymer adjuvant and Quil A immunostimulant. Since both Oralject™ formulations contained soybean and lima bean extracts, these data suggest that these two bean extracts are likely more important than white and red kidney bean extracts for intestinal delivery of NDV antigens in birds. It is anticipated that Oralject™ formulations can be specifically tailored, in part through the composition and concentration of lectin-containing bean extracts and adjuvant composition and immunostimulant type, to optimize the oral delivery of bioactive proteins to aquatic (e.g., salmonid) and terrestrial animals (e.g., birds).

### Example 10

### In vitro Anti-Protease Activity of Liquid Compositions

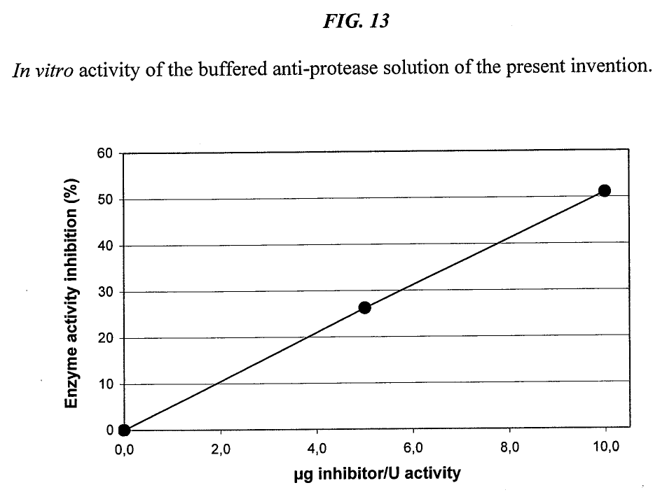

In certain embodiments, the compositions for oral delivery of bioagents may be formulated in a liquid form. In a non-limiting embodiment, 10 μl of enzyme extract is obtained from intestine of a fish, a fixed volume of the anti-protease liquid composition (see Example 11 below) and 500 μl of a Tris-HCL (50 mM)+CaCl₂ (10 mM) buffer (pH=7.5) are incubated for 60 minutes at room temperature. Afterwards, 500 μl of casein (0.5% v/w) in a Tris-HCl buffer (50 mM, pH=9) is added to the mixture and incubated for 30 minutes at room temperature. The reaction is stopped by the addition of 500 μl of trichloroacetic acid (20% v/w). The mixture is then incubated for 15 minutes on ice and 1 ml of the supernatant is put in a tube, centrifuged (13,000 rpm for 5 minutes) and the optical density read at 280 nm. Results from this experiment clearly show that the buffered anti-protease solution is effective in inhibiting the protease activity of the fish enzyme extract on the casein substrate (FIG. 13).

### Example 11

### Efficacy of a Liquid Composition to Reduce Blood Glucose Levels After Oral Administration of Insulin to Mice

Insulin is an essential hormone produced by the pancreas. Diabetes develops when the pancreas does not produce and secrete enough of this hormone, which is required for normal sugar, protein, and fat metabolism of the body. The potential usefulness of novel delivery systems for improving enteral absorption of poorly absorbed drugs, including peptide drugs such as insulin, has attracted considerable interest. Incorporation of insulin in the presently disclosed liquid composition can reduce tryptic digestion of insulin and enhance its enteral absorption.

In this study, the efficacy of a Formula 2 liquid composition to reduce blood glucose levels was evaluated after oral administration of insulin to mice.

### Material and Methods

Test System

| | |
|---|---|
| a) Species: | CD1 mice |
| b) Average weight: | 30 g |
| c) Number of animals: | 24 |
| d) Test article: | Human recombinant insulin (Sigma, #I2643) |

Liquid Composition

Preparation of protease inhibitor extracts. Protease inhibitors (4.44 g beans and 0.56 g albumen) were homogenized in Tris-HCL buffer (pH 7.5). The mixture was divided in equal volumes, centrifuged (3500 RPM for 10 minutes) and supernatants were pooled together. Supernatant was then concentrated by centrifugation through 10K cut-off Millipore filter tubes.

Preparation of uptake-increasing agents solution. In a 15 ml falcon tube, 1 ml of carbonate-bicarbonate buffer (0.25 M, pH 9.5) was added to 45 mg of sodium deoxycholate and stirred until complete dissolution. In another 15 ml falcon tube, 7 ml of carbonate-bicarbonate buffer (0.25 M, pH 9.5) was added to 450 mg of EDTA and stirred until complete dissolution. Both solutions were then pooled together.

Final preparation of liquid formulation. The final preparation of liquid formulation (Formula 2) was obtained by mixing the protease inhibitor extracts with the uptake increasing agents solution. The liquid formulation was then kept at 4° C. until use.

Experimental Design. Twenty four female CD1 mice of an average weight of 30 g were used in this study (see Table 14 for the identification of experimental groups). Mice were acclimated for at least 7 days prior to the beginning of the treatments. Mice were individually identified with a permanent ink marker on the tail of animals and were fasted overnight with water ad libitum prior to the beginning of the treatments.

CD1 mice were gavaged with insulin (200 U/kg) incorporated in a liquid composition (group A) and in water (group B). Negative control animals consisted of untreated mice (group C). At determined time periods post-administration (see Table 13), blood samples were taken via the retroorbital sinus and blood glucose levels were measured individually for each animal using an Ascencia™ DEX-2 blood glucose meter kit.

TABLE 14
Identification of experimental groups

| | | | Dose of | Volume | |
|---|---|---|---|---|---|
| | | No of | insulin | of | |
| # | Group | animals | U/kg | gavage | Blood samplings |
| A | Liquid composition | 8 | 200 | 300 μl | 0, 10, 20, 30, |
| | | | | | 50 min, 2, 4 h |
| B | Water | 8 | 200 | 300 μl | 0, 10, 20, 30, |
| | | | | | 50 min, 2, 4 h |
| C | Untreated control | 8 | — | — | 0, 10, 20, 30, |
| | | | | | 50 min, 2, 4 h |

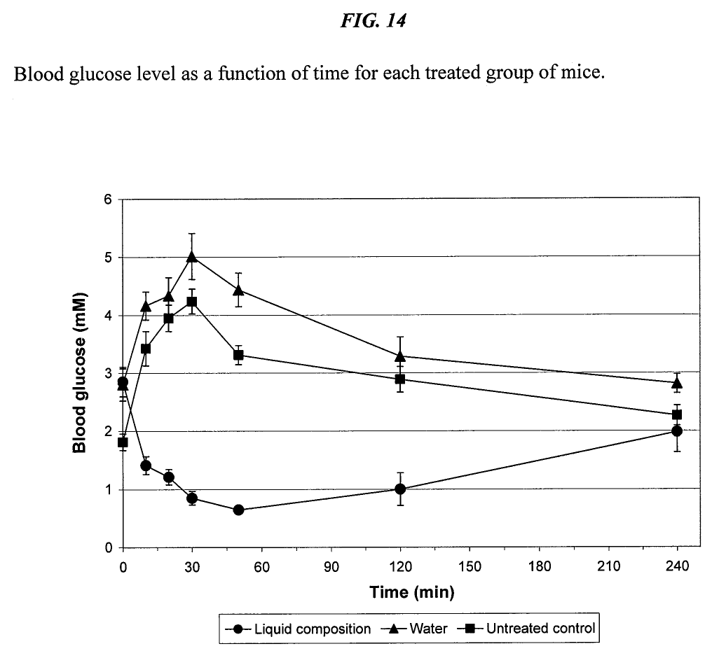

The results clearly show that the oral administration of a liquid composition (Formula 2) containing insulin markedly reduced blood glucose levels of mice, whereas animals treated orally with the same concentration of hormone in water showed no efficacy in reducing glucose levels (FIG. 14). These results further demonstrate that liquid compositions for the oral delivery of biotherapeutics (e.g., insulin) to terrestrial species can be improved through the inclusion of whole bean, pea, or other plant extracts, homogenates or ground powders that contain lectins (e.g., white and dark red kidney beans) and other immunomodulatory compounds such as fatty acids, isoflavones and saponins.

### Example 12

### Liquid Compositions for Delivery of Probiotics to Human Subjects

As discussed generally above, the field of probiotics involves delivery of living microorganisms to the gut, in part to attempt to balance the naturally occurring intestinal flora. Probiotic therapy is an area of rapidly increasing interest, although relatively few systematic studies of the effect of different formulations on the oral delivery of probiotic agents have been performed. In particular, it is likely that effective probiotic therapy may require delivery of large numbers of viable microorganisms to the large intestine.

A study was carried out to evaluate the ability of the liquid compositions of Example 11 to protect probiotics from destruction during transit through the digestive tract. The results disclosed below show that combining probiotic cultures with the disclosed liquid formulations significantly protected the viability of probiotics during passage though the upper gastrointestinal tract (stomach and small intestine), resulting in a 10,000-fold increase in viable bacteria reaching the lower intestine.

### Liquid Incubation Model

Methods

An overnight active culture (30 ml) of each tested organism was harvested at 7,000×g for 15 min at 4° C., washed twice with sterilized phosphate buffer saline (PBS, 0.01 M, pH 7.0), suspended in 30 ml of formula F1, F2 or F3 and stored at either 4 or 25° C. for 24 h. Control trials were also carried out by suspending the bacterial cells in 30 ml of PBS instead of the liquid formulas. Samples were taken at 0, 2, 4, 6, 8 and 24 h for the determination of viable count of microorganisms using de Man, Rogosa and Sharpe agar medium (MRS). Samples were 10-fold diluted in peptone water (0.15%, w/v) and appropriate dilutions were plated onto MRS agar. Plates were incubated anaerobically at 37° C. for 48 h.

Results

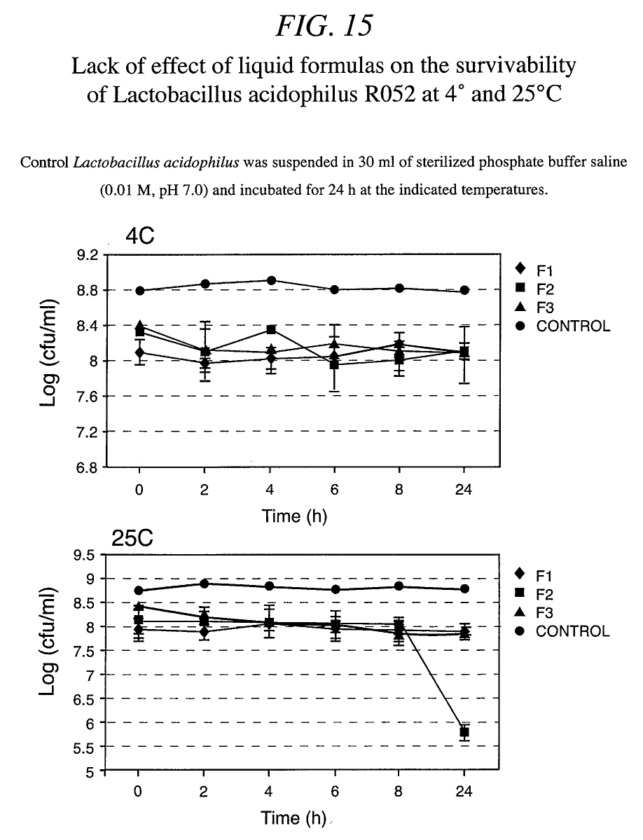

The results indicated that in a simple controlled temperature incubation model, the three formulas (F1, F2 and F3) did not affect the survivability of either B. thermophilum or Lb. acidophilus at either 4° or 25° C., except that a reduction of approximately 3 log cycles in viability of Lb. acidophilus was detected when the organism was incubated at 25° C. in the presence of F2 formula (FIG. 15).

### Gastrointestinal Dynamic Model (TIM-1)

Material and Methods

To provide a more realistic model for digestive tract degradation of probiotics, a previously developed and published computerized dynamic gastrointestinal model (TIM-1) was used. TIM-1 (TNO Pharma, 3700 A J Zeist, The Netherlands) is a multi-compartmental model which consisted of four compartments, including stomach, duodenum, jejunum and ileum. Each compartment is composed of two glass jackets holding a flexible membrane inside. The space between the membrane and the glass jackets is filled with water at 37° C. Mixing is achieved by alternately squeezing the flexible membrane through changing the pressure on the water. A temperature sensor and pH electrode are connected to each compartment. The levels in intestinal compartments (duodenum, jejunum and ileum) are monitored with sensors connected to each compartment.

To simulate gastric and intestinal secretions and to control pH in different compartments, the following solutions are continuously injected during the experiment and the volume of each fluid automatically recorded. HCl, pepsin and lipase were injected in the stomach. Bile, pancreatic juice, and bicarbonate were secreted into the duodenal compartment. Bicarbonate was secreted into the jejunal and ileal compartments to adjust the pH. The jejunal and ileal compartments were connected with hollow fiber devices that permit dialysis of the chyme.

Selective Media Evaluation

Prior to testing the survivability of Lb. acidophilus during the passage in the TIM-1 model, it was necessary to choose a selective medium for accurate determination of Lb. acidophilus viable counts. For this purpose, three media (at 43° C. for 72 h, under anaerobic conditions) were tested and compared with MRS agar medium (at 37° C. for 48 h, under anaerobic conditions) that is usually considered to be a reference medium for counting Lb. acidophilus and other lactic acid bacteria. This was in order to choose the most optimal selective medium that permitted recovering the maximum viable counts of Lb. acidophilus. The tested medium and incubation conditions were MRS, basal-maltose medium and basal-maltose medium.

Results indicated that basal-maltose medium at 43° C. for 72 h under anaerobic condition was the most effective medium and incubation conditions to recover Lb. acidophilus and counts determined on this medium did not differ from those determined on MRS agar at 37° C. for 48 h, under anaerobic condition.

Lactobacillus acidophilus Preparation

Briefly, 40 ml of an overnight active culture of Lb. acidophilus, grown in MRS broth medium, was harvested at 7,000×g for 15 min at 4° C., washed twice with sterilized PBS (0.1 M, pH 7.0), and suspended in 40 ml of the same buffer. For the control, sterilized PBS was added to the bacterial suspension and the total weight was adjusted to 300 g. The whole sample was stirred for 10 min at room temperature prior to injection into the TIM-1 model. For the liquid composition, 60 ml of the composition was added to the bacterial suspension, followed by stirring for 10 min, then the total weight was brought to 300 g using sterilized PBS and the whole mixture was further stirred for 10 min prior to injection into TIM-1 model.

Sampling and Calculations

The stomach compartment was sampled at 0, 45 and 90 min for the determination of viable counts of Lb. acidophilus. The duodenal compartment was sampled at 60, 120 and 180 min, and jejunal and ileal compartments were sampled at 60, 180 and 300 min.

Samples withdrawn from different compartments were 10-fold diluted in peptone water (0.15%, w/v) and appropriate dilutions were plated onto basal-maltose medium and plates were incubated anaerobically at 43° C. for 72 h. The survival of Lb. acidophilus was expressed as counts of colony forming unit/g sample (CFU/g). The sample weight was the amount of sample (300 g of sample injected into the stomach) that existed in different compartment at the sampling time.

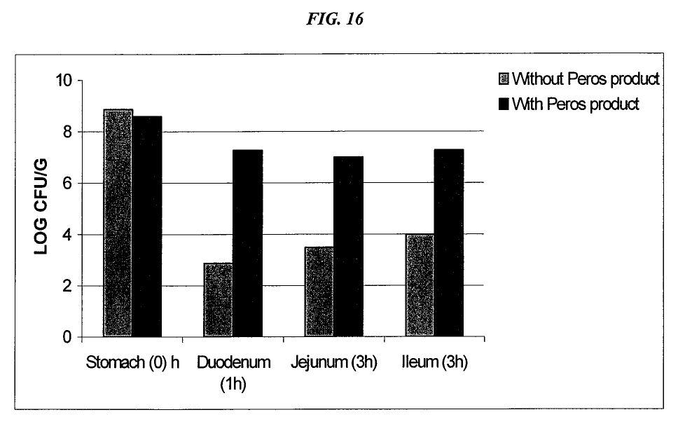

Results concerning the ability of Lb. acidophilus to survive gastrointestinal conditions in the TIM-1 model are given in Table 15 and FIG. 16. While little difference was observed in the stomach, it was obvious that the liquid composition substantially improved the survivability of Lb. acidophilus in duodenum, jejunum and ileal compartments. These results in a more realistic model system show that the liquid compositions may confer protection from harsh environmental conditions found in the gastrointestinal tract.

TABLE 15
Viability^{a} of Lactobacillus acidophilus R052 in absence and
presence of liquid composition.

| | In absence of liquid | | In presence of liquid | |
|---|---|---|---|---|
| Digestion | composition^{b} | | composition^{c} | |
| steps | CFU/g | % Survivability^{d} | CFU/g | % Survivability |
| Stomach | | | | |
| 0 min | 7.36 × 0⁸ ± 3.25 | 100 | 3.84 × 10⁸ ± 0.57 | 100 |
| 45 min | 2.29 × 10⁷ ± 0.03 | 3.11 | 2.24 × 10⁸ ± 0.24 | 58.3 |
| 90 min | <10² | — | 7.76 × 10³ ± 0.42 | 0.002 |
| Duodenum | | | | |
| 30 min | ND | — | 3.76 × 10⁸ ± 0.43 | 97.91 |
| 60 min | 7.49 × 10² | 0.0001 | 1.85 × 10⁷ ± 0.14 | 4.81 |
| 120 min | 1.85 × 10³ | 0.0002 | 6.47 × 10³ ± 1.78 | 0.001 |
| 180 min | <10² | — | 9.95 × 10³ ± 0.77 | 0.002 |
| Jejunum | | | | |
| 60 min | 3.75 × 10⁴ ± 4.56 | 0.005 | 1.28 × 10⁸ ± 0.35 | 33.3 |
| 180 min | 3.1 × 10³ | 0.0004 | 1.04 × 10⁷ ± 1.06 | 2.71 |
| 300 min | 2.51 × 10³ ± 2.12 | 0.0003 | 1.10 × 10⁷ ± 0.41 | 2.86 |
| Ileum | | | | |
| 60 min | 7.19 × 10⁴ ± 0.17 | 0.009 | 2.09 × 10⁸ ± 0.35 | 54.27 |
| 180 min | 9.91 × 10³ ± 2.82 | 0.001 | 1.88 × 10⁷ ± 0.27 | 4.90 |
| 300 min | 3.92 × 10³ ± 353 | 0.0005 | 1.27 × 10⁷ ± 1.03 | 3.30 |

^{a}viability was calculated as colony forming units (CFU) of viable Lb. acidophiuls per gram of sample existed in each compartment at different sampling time.
^{b}The tested sample contained 40 ml of an overnight culture of Lb. acidophilus (washed three times by phosphate buffer saline, 0.1M at pH 7.0 and then suspended in 40 ml of the same buffer) and the total weight was adjusted to 300 g using the same buffer prior to injection into TIM1 gastrointestinal model.
^{c}Sample contained 40 ml of an overnight culture of Lb. acidophilus (washed three times by phosphate buffer saline, 0.1M at pH 7.0 and then suspended in the same buffer), 60 ml of Peros product and the total weight was adjusted to 300 g using the same buffer prior to injection into TIM1 gastrointestinal model.
^{d}% survivability was calculated as viable count ((cfu/g sample)/(cfu/g determined at 0 min)) * 100

### Example 13

### Efficacy of a Solid Formulation to Orally Deliver Two Different Antibiotics to Aquatic Species

A study was carried out to determine the ability of a solid Oralject™ formulation (Formula 2, F2) to deliver orally high concentration of the antibiotics flumequine and flofenicol (FLOR®; Schering-Plough Corp., Lafeyette, N.J.) into plasma of Coho salmon (Oncorhynchus kisutch). Flumequine belongs to the fluoroquinolone group of antibiotics, possesses antimicrobial activity against gram-negative organisms and is primarily used in the treatment of enteric infections in food animals. Flofenicol belongs to the fenicol (chloramphenicol-like) group of antibiotics and possesses antimicrobial activity against gram-positive organisms.

In the first study, Coho salmon (n-70 fish/group) were treated with flumequine at a dose of 30 mg/kg incorporated in commercial feed or in formula F2. In the second study, fish were treated orally with florfenicol, antibiotic was given at 15 mg/kg incorporated in commercial feed or at 15 and 50 mg/kg in formula F2. In addition, florfenicol was also given via i.p. injection at 20 mg/kg. At predetermined time periods post-administration, blood samples were collected via the caudal vein according to routine procedures. Levels of antibiotics in plasma of fish were determined by high performance liquid chromatography (HPLC).

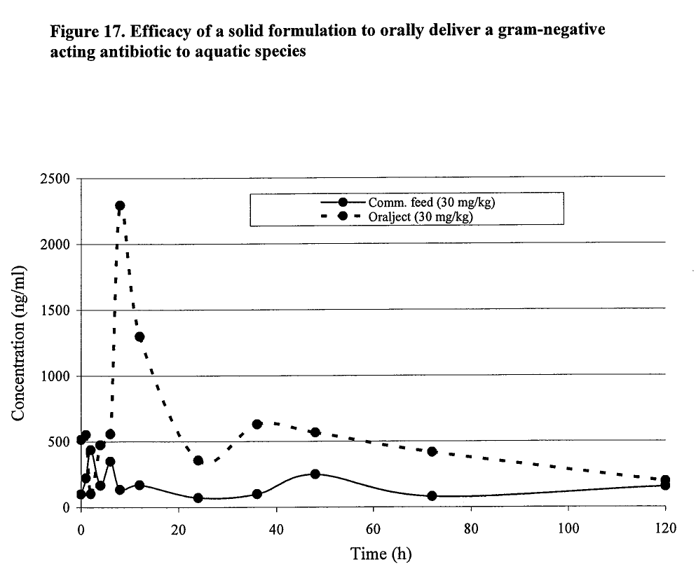

Results show that oral administration of flumequine (FIG. 17) in Formula 2 resulted in higher concentration in the plasma of fish when compared with fish fed with commercial feed containing antibiotic. Peak plasma concentration of flumequine was approximately 5-fold higher when delivered via Formula 2 when compared to delivery with commercial feed. In addition, plasma concentrations persisted for a longer time period when delivery in F2 compared to commercial feed control (FIG. 17).

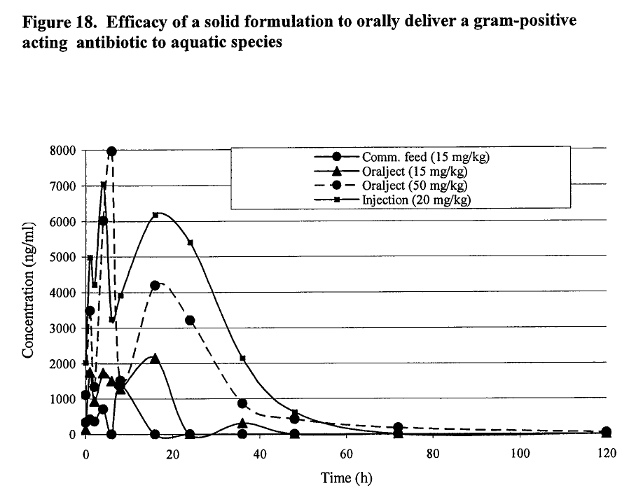

The results show that oral administration of florfenicol (FIG. 18) in Formula 2 resulted in higher concentration in the plasma of fish when compared with fish fed with commercial feed containing antibiotic. Results further show that the pharmakinetics of florfenicol was similar between F2 oral delivery and antibiotic injection.

The improved delivery of antibiotics in F2 compared to commercial feed delivery is further demonstrated through the pharmakinetic parameters of flumequine and florfenical. As shown in Table 16, the area under the curve (AUC) values of fish treated with flumequine and florfenicol incorporated in Formula 2 were 3.7 and 5.2 times higher, respectively, than values obtained for fish treated with the same concentration of antibiotics in commercial feed.

TABLE 16
Pharmakinetic parameters of flumequine and florfenicol administered
orally in either commercial feed or in Oralject ™ Formula 2 to
Coho salmon.

| | | Dose | Tₘₐₓ | Cₘₐₓ | AUC_{(0-120 h)} |
|---|---|---|---|---|---|
| Antibiotic | Administration | (mg/kg) | (h) | (μg/ml) | (μg h/ml) |
| Flumequine | Commercial | 30 | 8 | 2.29 | 16.8 |
| Flumequine | Oralject ™ (F2) | 30 | 2 | 0.44 | 61.9 |
| Florfenicol | Commercial | 15 | 8 | 1.38 | 6.7 |
| Florfenicol | Oralject ™ (F2) | 15 | 16 | 2.15 | 34.4 |
| Florfenicol | Oralject ™ (F2) | 50 | 6 | 7.96 | 136.0 |
| Florfenicol | Injection | 20 | 4 | 7.05 | 195.9 |

These results further demonstrate that solid compositions for the oral delivery of small drug molecules (e.g., antibiotics) to aquatic species can be improved through the inclusion of whole bean, pea, or other plant extracts, homogenates or ground powders that contain lectins (e.g., white and dark red kidney beans) and other immunomodulatory compounds such as fatty acids, isoflavones and saponins.

### Example 14

### Efficacy of a Solid Formulation to Orally Deliver a Viral Vaccine to an Aquatic Species

A study was conducted to evaluate the ability of the solid formulation (Formula 2, F2) to delivery orally an inactivated viral vaccine that can protect Atlantic salmon (2 g fish) against infectious pancreatic necrosis (IPN). Duplicate groups of Atlantic salmon (n=65/tank) were treated with F2 containing a commercially available IPN vaccine with an oral boost when they reached 15 g. Negative control fish remained untreated. All fish were challenged via injection of 0.1 ml of virulent IPN when they reached an average weight of 40 g. The efficacy of the different treatments to prevent infection was monitored from survival of fish as a function of time.

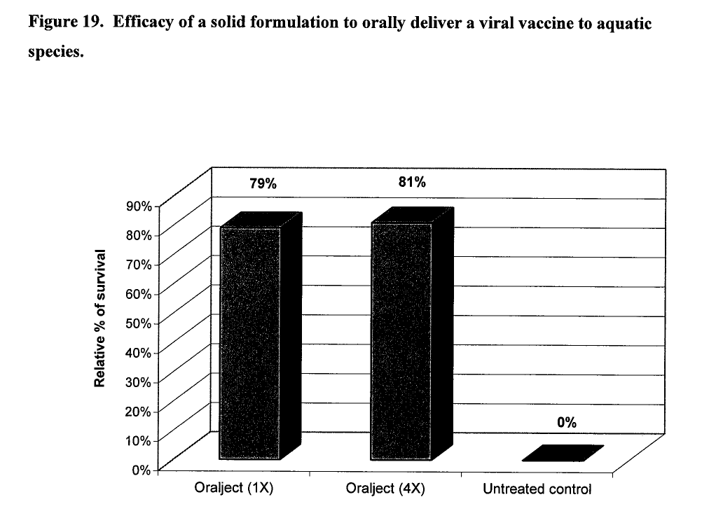

Results (FIG. 19) clearly demonstrate the efficacy of the solid formulation (F2) to protect small fish against infectious pancreatic necrosis. These results further show that immunoactive compositions for improved oral delivery of complex protein mixtures (e.g., whole, inactivated virus vaccine) can be improved through the inclusion of whole bean, pea, or other plant extracts, homogenates or ground powders that contain lectins (e.g., white and dark red kidney beans) and other immunomodulatory compounds such as fatty acids, isoflavones and saponins.

In final, the present Examples clearly show that methods and compositions for oral delivery of bioactive agents can be achieved depending on the desired indication (vaccine or therapeutic) and desired species (aquatic or terrestrial). The type and/or amount of naturally occurring ingredients, such as homogenates or fine ground powders of beans, peas, nuts, plant parts, fish meal or krill used in the claimed compositions can be rationally selected to optimize the content of specific lectins, isoflavones, polyunsaturated fatty acids, saponins and/or protease inhibitors present in the final composition.

The methods and compositions are effective for oral delivery of a wide variety of bioactive agents to a wide range of subjects. Methods and compositions for oral delivery of bacteria and viral vaccines comprised of larger molecular weight complex biological mixtures are different from those for oral delivery of therapeutics comprised of smaller molecular weight, less complex, and relatively pure mixtures. Methods and compositions for oral delivery of vaccines and bioactive agents with increased immunostimulatory, increased immunotolerant or increased immunosuppressive activity can be achieved.

For oral delivery of bacterial and viral vaccines in solid or liquid formulations in aquatic species, specific lectins such as ConA or PHA, and specific fatty acids such as N-6 PUFAs are preferentially used. For oral delivery of bacterial and viral vaccines in solid or liquid formulations in terrestrial species, the addition of specific lectins such as LBL and SBA can further improve the immune response. For oral delivery of bacterial and viral vaccines in aquatic and terrestrial species in solid or liquid formulations in which a cell-mediated, Th1 response is desired, relatively low amounts of isoflavone and relatively high amounts of Group B saponins or sapogenol are used. For oral delivery of bacterial and viral vaccines in solid or liquid formulations in aquatic and terrestrial species in which a cell-mediated, Th2 response is preferred, relatively high amounts of isoflavone and relatively low amounts of saponins are used. Oralject™ formula 1 is preferred for administration of vaccines to avian species, whereas Oralject™ formula 2 or formula 7 is preferred for administration of vaccines to aquatic species and mammalian terrestrial species.

Oral delivery of biotherapeutic agents in solid or liquid formulations comprised of smaller molecular weight, less complex, and relatively pure mixtures to aquatic and terrestrial species can be improved through the inclusion of whole bean, pea, nut or other plant extracts, homogenates or ground powders that contain lectins (e.g., white and dark red kidney beans) and other immunomodulatory compounds such as fatty acids, isoflavones and saponins. The concentration of the lectins, fatty acids, isoflavones and saponins can be adjusted to be immunostimulatory, immunotolerant or immunosuppressive. For example, for slower, more sustained bioavailability of the biotherapeutic agent, a formula which is immunotolerant or immunosuppresive is preferred so that the formulation is not rapidly cleared by the immune response. Oralject™ formula 2 is preferred for administration of biotherapeutic agents in aquatic species and mammalian terrestrial species.

All of the COMPOSITIONS and METHODS disclosed and claimed herein can be made and executed without undue experimentation in light of the present disclosure. While the compositions and methods have been described in terms of preferred embodiments, it is apparent to those of skill in the art that variations maybe applied to the COMPOSITIONS and METHODS and in the steps or in the sequence of steps of the methods described herein without departing from the concept, spirit and scope of the invention. More specifically, certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

All documents, or portions of documents, cited in this application, including but not limited to patents, patent applications, articles, books, and treatises, are expressly incorporated herein by reference in their entirety.

Buttle L G, Burrells A C, Good J E, Williams P D, Southgate P J, Burrells C. 2001. The binding of soybean agglutinin (SBA) to the intestinal epithelium of Atlantic salmon, Salmo salar and rainbow trout, Oncorhynchus mykiss, fed high levels of soybean meal. Vet Immunol Immunopathol. 80(3-4):237-44.

Choi, C., Cho, H., Park, J., Cho, C., Song, Y. 2003. Suppressive effects of genistein on oxidative stress and NFkB activation in RAW 264.7 macrophages. Biosci. Biotechnol. Biochem. 67(9):1916-1922.

Cooke P S, Selvaraj V, Yellayi S. 2006. Genistein, estrogen receptors, and the acquired immune response. J Nutr. 136(3):704-708.

Gauthier, D. T., Cartwright, D. D., Densmore, C. L., Blazer V. S., Ottinger, C. A. 2003. Measurement of in vitro leucocyte mitogenesis in fish: ELISA based detection of the thymidine analogue 5′-bromo-2′-deoxyuridine. Fish Shellfish Immunol. 14:279-288.

Henricks, H. G., Koninkx, J. F., Draaijer, M., van Dijk, J. E., Raaijmakers, J. A., Mouwen, J. M. 1987. Quantitative determination of the lectin binding capacity of small intestinal brush-border membrane. An enzyme linked lectin sorbent assay (ELISA). Biochim Biophys Acta. 905(2):371-375.

Jung, H. J., Kim, S. G., Nam, J. H., Park, K. K., Chung, W. Y., Kim, W. B., Lee, K. T., Won, J. H., Choi, J. W., Park, H. J. 2005. Isolation of saponins with the inhibitory effect on nitric oxide, prostaglandin E₂, and tumor necrosis factor-alpha production from Pleurospermum kamtschaticum. Biol. Pharm. Bull. 28(9):1668-1671.

Jung K S, Ahn M J, Lee Y D, Go G M, Shin T K. 2002. Histochemistry of six lectins in the tissues of the flat fish Paralichthys olivaceus. J Vet Sci. 3(4):293-301.

Kilpatrick, D. C., A. Pusztai, G. Grant, C. Graham, S. W. Ewen. 1985. Tomato lectin resists digestion in the mammalian alimentary canal and binds to intestinal villi without deleterious effects. FEBS Lett 185:299-305.

Kjaer, T. M., and Frokiaer, H. 2002. Modulation of ovomucoid-specific oral tolerance in mice fed plant extracts containing lectins. Br. J. Nutr. 88(6):671-680.

Knudsen D, Ron O, Baardsen G, Smedsgaard J, Koppe W, Frokiaer. 2006. Soyasaponins resist extrusion cooking and are not degraded during gut passage in Atlantic salmon (Salmo salar L.). J Agric Food Chem. 54(17):6428-35.

Koninkx, J. F., H. G. Hendriks, J. M. van Rossum, T. S. van den Ingh, J. M. Mouwen. 1992. Interaction of legume lectins with the cellular metabolism of differentiated Caco-2 cells. Gastroenterology 102:1516-1523.

Leavitt, R. D., Felsted, R. L., Bachur, N. R. 1977. Biological and biochemical properties of Phaseolus vulgaris isolectins. J Biol. Chem. 252(9):2961-2966.

Lis, H., and Sharon, N. Lectins: Their chemistry and application to immunology. In: The Antigens, edited by M. Sela. New York: Academy Press, 1977, vol 4, pp. 428-529.

Nowell, P. C. 1960. Phytohemagglutinin: an initiator of mitosis in cultures of normal human leukocytes. Cancer Res., 20:462-466.

Rachon D, Rimoldi G, Wuttke W. 2006. In vitro effects of genistein and resveratrol on the production of interferon-gamma (IFNgamma) and interleukin-10 (IL-10) by stimulated murine splenocytes. Phytomedicine. 13(6):419-24.

Ruddon, R. W., Weisenthal, L. M., Lundee, D. E., Bessler, W., and Goldstein, I. J. 1974. Stimulation of mitogenesis in normal and leukemic human lymphocytes by divalent and tetravalent lima bean lectins. PNAS. 71(5):1848-1851.

Ryder, S. D., J. A. Smith, J. M. Rhodes. 1992. Peanut lectin: a mitogen for normal human colonic epithelium and human HT29 colorectal cancer cells. J. Natl. Cancer Inst 84:1410-1416.

Sage H J, Yates L D, Horton C B. 1982. Binding of soybean agglutinin to glycolipid components of porcine lymphocyte plasma membranes. Arch Biochem Biophys. 216(2):685-92.

Tang, S., Li, D., Qiao, S., Piao, X., Zang, J. 2006. Effects of purified soybean agglutinin on growth and immune function in rats. Arch Anim. Nutr. 60(5):418-426.

Zhang P, Smith R, Chapkin R S, McMurray D N. 2005. Dietary (n-3) polyunsaturated fatty acids modulate murine Th1/Th2 balance toward the Th2 pole by suppression of Th1 development. J Nutr. 135(7):1745-51.

Zhang R, Li Y, Wang W. 1997. Enhancement of immune function in mice fed high doses of soy daidzein. Nutr Cancer. 1997;29(1):24-8.

Zou J, Carrington A, Collet B, Dijkstra J M, Yoshiura Y, Bols N, Secombes C. 2005. Identification and bioactivities of IFN-gamma in rainbow trout Oncorhynchus mykiss: the first Th1-type cytokine characterized functionally in fish. J Immunol. 175(4):2484-94.

What is claimed is:

1. A composition for oral delivery of a bioactive agent to a subject comprising:
a) at least one lectin;
b) at least one isoflavone c) at least one polyunsaturated fatty acid; and
d) at least one saponin;
wherein the composition is effective for oral delivery of a bioactive agent to a subject.

2. The composition of claim 1, further comprising at least one protease inhibitor.

3. The composition of claim 2, further comprising at least one surfactant.

4. The composition of claim 3, further comprising a buffer.

5. The composition of claim 1, wherein the at least one lectin, isoflavone, polyunsaturated fatty acid and saponin are contained in one or more finely ground powders, homogenates or extracts from a plant or animal source.

6. The composition of claim 5, wherein the animal source is fish meal or krill.

7. The composition of claim 5, wherein the plant source is bean, pea or nut.

8. The composition of claim 7, wherein the bean, pea or nut is a soybean, lima bean, fava bean, kidney bean, red kidney bean, broad bean, jequirity bean, jack bean, small pea (Pisum sativum), sweet pea, Rosemary pea, lentil, vetch or peanut.

9. The composition of claim 5, wherein the finely ground powder has a particle size of about 0.1, 0.2, 0.25, 0.3, 0.4, 0.5, 0.6, 0.7, 0.75, 0.8, 0.9, 1.0, 1.5, 2.0, 2.5, 3.0, 4.0 or 5.0 millimeters or any range in between.

10. The composition of claim 5, wherein at least one lectin, isoflavone, polyunsaturated fatty acid or saponin is added to the one or more extracts or homogenates.

11. The composition of claim 7, further comprising two or more extracts or homogenates of bean, pea or nut.

12. The composition of claim 11, wherein the proportions of different finely ground powders, extracts or homogenates in the composition are selected to optimize oral delivery of the bioactive agent.

13. The composition of claim 11, wherein the proportions of different finely ground powders, extracts or homogenates in the composition are selected to optimize stability, shelf life, or delivery to the oral compartment of the bioactive agent.

14. The composition of claim 11, wherein the particle sizes of the finely ground powders are selected to optimize stability, shelf life, or delivery to the oral compartment of the bioactive agent.

15. The composition of claim 1, further comprising one or more bioactive agents.

16. The composition of claim 12, wherein the selection is based on the lectin, isoflavone, fatty acid and saponin contents of the extracts or homogenates.

17. The composition of claim 12, wherein the selection is further based on the species of the subject to whom the composition is to be orally administered.

18. The composition of claim 2, wherein the at least one protease inhibitor is contained in at least one extract or homogenate of bean, pea or nut.

19. The composition of claim 1, wherein the at least one lectin is selected from the group consisting of SBA, PHA-E, PHA-L, LBL, LCA, LOA, Con A, PSA and PNA.

20. The composition of claim 1, wherein the at least one saponin is selected from the group consisting of soyasaponin A(1), soyasaponin A(2), soyasaponin I, soyasaponin B, deacetylated soyasaponin, acetylated soyasaponin, soyasaponin II, soyasaponin III and soyasapogenol B monoglucuronide.

21. The composition of claim 1, wherein the at least one fatty acid is selected from the group consisting of soybean n-3, soybean n-6, kidney bean n-3, kidney bean n-6 and lima bean n-6 polyunsaturated fatty acid.

22. The composition of claim 1, wherein the at least one isoflavone is selected from the group consisting of gentisein, daidzein and biochanin A.

23. The composition of claim 1, wherein the subject is a human, primate, fish, trout, salmon, carp, shrimp, bird, aves, chicken, duck, cow, bovine, pig, sheep, ovine, goat, caprine, dog, canine, cat, feline, horse, equine, alpaca, camelid, or llama.

24. The composition of claim 15, wherein the bioactive agent is selected from the group consisting of drugs, pharmaceuticals, toxins, anti-cancer agents, anti-inflammatory agents, antibiotics, antifungals, antiviral agents, anti-parasitic agents, vaccines, adjuvants, antigens, hormones, growth factors, cytokines, chemokines, immunomodulators, interferons, interleukins, hematopoietic factors, coagulation factors, anti-angiogenic factors, pro-apoptosis factors, neurotransmitters, neuromodulators, enzymes, agonists, antagonists, antibodies, antibody fragments, fusion proteins, proteins, polypeptides, peptides, nucleic acids, lipids, polysaccharides, carbohydrates and steroids.

25. The composition of claim 17, wherein the subject is a fish, the bioactive agent is a vaccine, and the powders, extracts or homogenates are selected to contain low (about 0.1 to 0.5 μg/ml) levels of ConA and/or PHA.

26. The composition of claim 17, wherein the subject is a bird, the bioactive agent is a vaccine, and the powders, extracts or homogenates are selected to contain moderate (about 1 to 2 μg/ml) levels of ConA and/or PHA.

27. The composition of claim 17, wherein the subject is a terrestrial mammal, the bioactive agent is a vaccine, and the powders, extracts or homogenates are selected to contain high (about 5 μg/ml) levels of ConA and/or PHA.

28. The composition of claim 17, wherein the subject is a pig or cow, the bioactive agent is a vaccine, and the powders, extracts or homogenates are selected to contain about 0.1 μg/ml PHA.

29. The composition of claim 17, wherein the agent is a bioactive agent other than a vaccine, and the powders, extracts or homogenates are selected to not contain mitogenic lectins.

30. The composition of claim 17, wherein the bioactive agent is a vaccine, the subject is a fish or bird, and the composition is Formula 2; or the the bioactive agent is a vaccine, the subject is a terrestrial mammal, and the composition is Formula 2 or Formula 7.

31. The composition of claim 17, wherein the agent is a bioactive agent other than a vaccine, and the composition is Formula 1.

32. The composition of claim 17, wherein the bioactive agent is a vaccine and the powders, extracts or homogenates comprise white or dark red kidney beans.

33. The composition of claim 17, wherein the agent is a bioactive agent other than a vaccine and the powders, extracts or homogenates are selected to exclude white or dark red kidney beans.

34. The composition of claim 17, wherein the agent is a vaccine and the powders, extracts or homogenates are selected to include N-6 PUFAs or wherein the agent is a bioactive agent other than a vaccine and the the powders, extracts or homogenates are selected to include N-3 PUFAs.

35. A method of oral delivery of a bioactive agent to a subject comprising:
a) obtaining a composition according to claim 17, further comprising one or more bioactive agents;
b) orally administering the composition to a subject.

36. The method of claim 35, wherein the agent is a vaccine and the composition comprises between about 10 to 100 nM genistein or daidzein to generate a T-helper 2 response, or about 1 μM genistein, daidzein or biochanin to generate a T-helper 1 response.

37. The method of claim 35, wherein the agent is a vaccine and the composition comprises high concentrations (about 100 μg/ml) of sapogenol B or Group B saponins to suppress T-helper 2 response and induce T-helper 1 response.
